(19) United States
(12) Patent Application Publication
(10) Pub. No.: US 2019/0054347 A1
(43) Pub. Date: Feb. 21, 2019

(54) WEARABLE SPORTS GUIDANCE COMMUNICATION SYSTEM AND DEVELOPERS TOOL KIT

(71) Applicants: Michael Saigh, Clayton, MO (US); Xiaohong Zhang, Clayton, MO (US); Susan Carpenter, Clayton, MO (US)

(72) Inventors: Michael Saigh, Clayton, MO (US); Xiaohong Zhang, Clayton, MO (US); Susan Carpenter, Clayton, MO (US)

(21) Appl. No.: 16/169,520

(22) Filed: Oct. 24, 2018

Related U.S. Application Data
(63) Continuation-in-part of application No. 14/754,691, filed on Aug. 18, 2015.

(51) Int. Cl. A63B 24/00; A61B 5/00; A61B 5/103

(52) U.S. Cl. CPC A63B 24/0062; A61B 5/682; A61B 5/103; A61B 5/745; A63B 2024/0081; A63B 2230/00; A63B 2220/00; A63B 2225/50; A63B 2024/0065

(57) ABSTRACT

A sports training and guidance platform network which intertwine various wearables is provided. The network includes at least one wearable containing one biosensor worn on the body, and input from a data stream, analytics and accessed on mobile devices. According to this aspect, the wearable network will measure and compare individual or team performance with various biosensor data, and motion i.e., accelerometers, gyroscopes. A sports training toolkit which includes the wearable communication network and platform and system is also provided.

### RELATED APPLICATIONS

This application is a Continuation-in-Part of U.S. patent applicatio Ser. No. 14/754,691 entitled “INTEROPERABLE WEARABLE TECH DEVICES SYSTEM AND COMMUNICATION PLATFORM” which was filed on Aug. 18, 2015. This previous patent is incorporated herein by reference.

The present disclosure relates generally to smart, oral sensor devices and the integration of such with mobile communications, alerting and related technologies for both animals and humans, referred to herein as an WEARABLE SPORTS GUIDANCE COMMUNICATION SYSTEM AND DEVELOPERS' TOOLKIT.

### BACKGROUND

Wearable devices (such as devices produced by Fitbit, Inc. and others) exist for tracking activity and basic fitness metrics. These devices, which are designed for individual, fitness-conscious users such as casual or recreational athletes, measure data such as a number of steps walked, heart rate, quality of sleep, steps climbed, and other personal metrics.

### SUMMARY OF THE INVENTION

These wearable devices are not only inaccurate but are not designed for serious athletes such as players of professional, Olympic, college and high school sports, including all land sports, ball sports, and water sports.

In addition, current technology and current product designs are limited and do not account for the measurement of the player's physiological characteristics compared to the player's performance, nor do the devices measure and/or analyze physiological characteristics and performance for teams (e.g. comprising more than one athlete).

At the same time, biosensor sampling involves simple and non-invasive collection methods which allow easy and fast diagnostic testing. For example, oral cavities contain salivary secretions, an abundant blood supply, lymph nodes, ingested pathogens, ingested toxins, ingested allergens, ingested drugs, ingested nutrients, and/or ingested food constituents. Biosensors located on, in, or near parts of the body of the player, including the oral cavity, chest, ear, mouth, eye, neck, face, leg, arms, back, and/or foot, among other examples, can be networked, and the biosensor data can be compared to performance data for players and/or teams.

The presence of various biomarkers permits accurate reflection of normal and disease states in animals and humans. Information derived from the oral cavity is capable of augmenting, or possibly replacing blood sampling, and/or oral cavity information may be used as an efficient precursor before other more invasive medical diagnostics are employed. However, currently available methods for the detection of various biomarkers are inefficient and do not alert or communicate information derived from biomarkers contained when networked in a rapid manner. Currently a network of biosensors, sensors, and devices which measure activity are not capable of providing biosensor data that would be useful to or even required by coaches, trainers, players and managers of serious individual and/or team sports.

Thus, there is a need for a sports wearable network designed for serious athletes, for accurate health information gathering, assessment, monitoring, and ultimately, improved athletic performance, training, assistance and intervention, for example, when players' biomarkers are beyond a safe zone or optimal performance zone.

In addition, currently there is a profound lack of integration between a multitude of cross-linked technologies and skills when determining information regarding metadata diagnosis; with geometric tracking, multimedia, communication networks, analytics, alerting, and kinematics for individuals, team sports, organizational groups, animals and humans, which enhance health and performance. In addition, these current limitations restrict a multi-dimensional approach which could seamlessly measure individuals and animals with greater accuracy, convenience, yet far less intrusively. In addition, the lack of integration between disciplines fails to address the growing need for the next level of metadata and biological tools which could provide early detection of an athlete's health, early warning signs of dehydration, heart problems, past concussions, and other medical issues. Furthermore, the lack of integration of bio-stats when compared to players' or teams' performance does not balance an athlete's skills with their real-time health. Thus, coaches today lack necessary information for preparing and implementing tailored training programs for players and athletes that balance training and fitness with exertion and physical limitations. The current invention balances both performance with physical limitations for both humans and animals (e.g. racehorses).

The present invention provides smart wearable devices, systems and methods relating thereto, as well as auxiliary devices and methods, for greatly improving animal and human well-being, sports performance and physiological set-points through innovations in such technologies. The invention combines its enhanced, “smart”, sensor devices and methods with communications, software management, data management, instant and long-term animal and human analyses, multimedia inputs, visualizations, geometric motion, tracking, kinematics, alerting, therapeutic, electronic medical records and other beneficial systems not previously available.

The Wearable Sports System (WSS) of the invention provides for communication systems and alerting technology that link a multitude of biological information inputs together. This method of gathering biological information from wearable devices provides the basis for a real-time or near-time snapshot of an animal or human's optimal sports performance and physical limitations.

Accordingly, a sensor alerts and communication system, and methods and devices related to and used in conjunction therewith are provided which address the needs and provide the advantages outlined herein.

Also the present invention provides a Sports Guidance Technologies (SGT) device in which sensors are networked together in response to alerts and/or signals from the wearable sports system.

In an aspect of the invention, a device is provided which includes a smart sensor receptacle for a sensor. SGT embedded wearable sensors could be utilized in various contexts including, but not limited to, high level sports performance, animal sports and recreational performance, and other medical diagnostics, and analytics function. The device includes one or more sensors contained within or upon the receptacle or multiple receptacles networked and communicated to mobile device (smartphone, tablet, etc.) used for example by trainers and coaches or the athletes themselves.

In another embodiment of the invention, the wearable sports system can streamline and integrate performance measurements such as, but not limited to, various geometric models, visualizations, complex spatial-temporal relations, human and animal facial and physical relationships (individually and group), data associations (i.e., pixels, auditory, motion, optimum breathing, oral air-flow, accelerometers, accelerometer arrays, tri-axial accelerometers, gyroscopes, tri-axial gyroscopes, pressure sensors, magnetometers, goniometers, metabolic biosensors, high-definition video capture, body-wearable sensors, RFIDs, readers, positioning, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple, various degrees of dynamic alerting, tracking, positioning, multi-media, analytics, historical and other comparative data inputs, communications and platforms etc.). Collectively, these inputs can be synced and integrated with all forms of data capture. The wearable sports system can provide important real-time or near time analytics in order to correct or modify motions and behaviors for individuals, team sports or organizational groups for animals and humans.

In a further embodiment, the invention provides a wearable sports system including the above-described smart receptacle, one or more sensors contained within, attached, or upon the receptacle and at least one interface with a network configured to utilize the information obtained from the one or more sensors.

It is understood by anyone familiar with the art that independent to wireless storage, the data could be stored in any SGT device through any digital storage device, connector, or mechanism.

The invention provides, in another embodiment, a system which includes a device configured to be inserted or attached to an animal or human. The device includes a smart sensor receptacle for one or more sensors wherein the receptacle is selected and could be customized for any human or animal condition. For example, the receptacle can be selected from the group consisting of a horse-bit, a thermometer, a receptacle configured so that it cannot be swallowed, a receptacle for babies or adults with biosensors on one side and a RFID on the other side which is on the outside of a mouth, a customized teeth retainer which could be attached to a sports guard to enhance functionality and purpose, a receptacle to be attached to a human or animal body, an insert in a gum, an attachment to socks, shoes, hats, wristbands, headbands, helmets, goggles, ear modules, clothing, eyewear, etc. SGT device can include any combination of biosensors and RFID tags, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in the mouth or integrated outside of a mouth. In addition, other consumer products could include a subscription database with software analytics which measure a player's performance as it matches and relates to his or her physiological analysis.

In yet a further embodiment of the invention, a method is provided for obtaining sensor data from a human and/or an animal. The smart receptacle contains or receives within or upon it one or more sensors capable of providing information relevant to the health or a physiological characteristic of the human or animal. The method further involves activating or monitoring the one or more sensors to obtain or analyze the information relevant to the health or a physiological characteristic of the human or animal and transmitting at least some portions of the health or physiological information or analysis to a network capable of utilizing the information obtained.

The recognition component in these systems and methods of the invention, often called a receptor, can use, e.g., biomolecules from organisms or receptors modeled after biological systems to interact with an analyte of interest. This interaction can be measured by a biotransducer which outputs a measurable signal proportional to the presence of a target analyte in the sample.

In another aspect of the method of the invention, the receptacle used in the above method includes a smart sensor receptacle for one or more sensors for example, but not limited to, a retainer combination sports guard, an attachment to a tooth, an attachment to an animal or human body, an insert in a gum, socks, shoes, hats, wristbands, headbands, helmets, goggles, ear modules, clothing, eyewear, etc., inserts with biosensors, sensors, communication capabilities including but not limited to camera, audio, thermal IR, multi-media, speakers, a RFID, etc. on the inside or outside of a mouth and an animal toy which is configured not to be swallowed, securely and strategically placed touching a body or within an animal's or human's oral cavity, eye cavity, ear cavity and nose cavity.

In yet an additional aspect, the invention includes a wearable sports system for an animal or human. The wearable sports system includes a smart, wearable or attachable device. The smart, wearable, attachable or externally insertable device is configured to obtain information from, provide information to, or both, the one or more sensors located on the body or within the aforementioned cavity receptacle. And, the one or more sensors or the smart, external device, or both, are configured to transmit the information to a network.

Also provided is a customizable development tool kit or platform for multiple SGT purposes and functions and for building a wearable sports system to provide information, analysis or alerts for an animal, animals, human or humans, comprising a kit or platform of customizable components to meet the needs of a developer, consumer or user of the system, the components comprising at least one sensor inserted or attached to the animal, animals, human or humans, at least one receptacle configured to contain or receive the sensor, and at least one network unit configured to receive information, analysis or alerts from or transmit information, analysis or alerts to the at least one sensor and analyze, transmit, or both, the information, analysis or alerts obtained or received, wherein components for selecting the sensor receptacles, the sensors, and the network units are made available to the developer, consumer or user to construct or have constructed a system configured to obtain or transmit information, analysis or alerts customized to meet the specific needs of the developer, consumer or user.

In addition, the SGT device could utilize the network of wearable devices to guide and train individuals or teams. For example, a vibration on the upper right arm when a player needs to pass the ball to another player to the right side of him. Coaches and trainers could manually activate one or more vibrations or other mechanisms to signify directions or signals, ball handling and an athlete's timing and mechanics. Furthermore, the coach or trainer could distinguish for example the strength of the vibration or location of the vibration to signify the movement of a player, rotation, arm movement, ball, bat, hockey stick in any direction.

In yet another embodiment, the SGT network could activate one or more wearables not only to define a player's exact motion but also to correct the player's motor skills and make adjustments when needed to optimize a player's or teams' performance.

In yet another embodiment, wearable sports system can be employed to compare the performance and kinematics of an individual player with the advanced player in order to pinpoint the areas of development for the individual. For example, back-hand stroke angular motion and stroke power could be greater in advanced tennis players due to their use of efficient kinetic chains.

In addition, automatic SGT artificial intelligence rather than the coach could be customized to help directionalize the player's arm movement when throwing a ball, catching a ball or for any and all sports activity. For example, the SGT artificial intelligence could analyze and scan a player's body and body parts. The system can determine the most efficient motion for the player when pitching a fast ball for example and correct or adjust his motion through the vibration or tightening the wearable to help direct the muscles needed to throw the ball. Visualizing the exact movements of a golf swing for example through virtual three dimensional images can help translate it into reality for the player. All sports have optimal motion and optimal mechanics which are refined through repetitive training sessions. In one embodiment of the present invention wearables could assist and guide an athlete whether in an individual sport or a team sport.

In another embodiment of this invention, any type of robotics, including, but not limited to, airborne, water, land robotics and others can be used in sports training. For example, GPRS drone locators can be placed in the practice vicinity (air, water and land etc.), and can film, monitor, track and guide each player on the field through the wearables that the player puts on. Robotic systems can function as coaches, trainers, players or assistants, etc. A portion of the body of the robotics (arms, arm sleeves, leg sleeves, head, skull, face, upper-back, lower back, legs, knees, shoulder, elbow, hip, ankle, armpit, hand, glove, foot, toe etc.) can also be employed in training. For example, robotic sleeves with embedded artificial intelligence which automatically calculates the angle, velocity and strength, etc. of shooting based on the physical characteristics of the basketball player can be used to train shooting and improve the free throw percentage.

In another embodiment of this invention, coaches or trainers are replaced by a software program or artificial intelligence. Data from wearables, sensors on sports equipment, environmental sensors, and data entered about the athletes' health and historical performance data could be used to assist in training. This could enable athletes training and increase their skills when trainers are not available.

In another embodiment of this invention, the SGT device functions as a coach and trainer to enhance an athlete's performance. Smart clothing and smart equipment assist in determining exact movement, strength, bounce, throw, etc. This smart clothing and equipment could further assist in determining how to improve any athlete's performance and act as a guide, coach, or trainer. This smart coach could guide by use of all physiological senses and perceptions including ophthalmoception, audioception, gustaoception, olfacoception or olfacception, tactioception, (thermoception), kinesthetic sense (proprioception), pain (nociception), balance (equilibrioception), vibration (mechanoreception), and various internal stimuli (e.g. the different chemoreceptors), tension sensors, pressure, stretch receptors, time perception and other beneficial systems not previously available. The intensity of these senses and perceptions input could be used to guide differently.

In yet another embodiment of the present invention, wearable devices are used by the player to adapt to environmental conditions such as noise level, humidity, altitude, environmental temperature, precipitation, humidity, distance, wind speed and direction, hill slope and height, soil and sand conditions, grain, grass type and height, icy conditions, raining conditions, slippery conditions etc. by adjusting the player's movements, for example, to take smaller more deliberate steps or pass the ball further in response to a 10 mile an hour wind from the northwest (NW). The SGT device could calculate and logistically guide the player to adjust his or her pass, hit or kick to counter the wind factor or any weather related or environmental conditions.

In another embodiment of the present invention, artificial intelligence guides one or more players through a combination of kinematics, high definition video, animation, facial and body recognition to determine precision movements and the exact measurement of a player's touch of a ball for example.

In another embodiment of the present invention, the convergence of wearable technologies enables coaches and referees to better determine fouls when video footage is not taken at the right angle or angles and enables coaches to review computer animation and precise movement as it relates to other players, logistics and precision location.

In yet another embodiment of the present invention the wearable device(s) could contain impact sensors, motion sensors, gyroscopes, tri-axial gyroscopes, accelerometers, accelerometer arrays, tri-axial accelerometers, pressure sensors, magnetometers, goniometers and XYZ locators to determine the player's precise location on the sports field. These wearables can be positioned at or on all parts of the athlete's body through the SGT device to detect exact movement on the location for example of the arm or arms or any other body part.

In another embodiment of the present invention, wearable sports system (WSS) which networks all body sensors can be used to estimate whole body center of mass, whole body velocity and acceleration real time or near time in the field with full body modeling. For example, when the acceleration of the whole body center of mass is measured, phases of the stroke cycle in which propulsive forces are not being applied effectively and the body encounters great resistance can be identified and linked to the technique of the swimmer to improve performance.

In another embodiment of the present invention, wearable sports system are applied to quantify an individual's movement patterns during athletic maneuvers in order to increase the probabilities of identifying those at increased risk of injuries.

In another embodiment of the present invention, kinematic data obtained using the SGT device can assist in the choice of equipment such as balls, bats, rackets, clubs and tees, etc. For example, there are different types of racket which vary in mass, swing weight and twist weight etc. Utilizing different types of racket could result in changes in shoulder joint power, internal/external rotation peak moments, and activities in latissimus dorsi muscles etc. during acceleration and follow through phase. This information is essential to quantify the loads on the body during play in order to improve the performance and reduce injuries.

In another embodiment of the present invention, sensors are embedded in balls, hoops, bats, rackets, clubs and tees, etc. to precisely determine movement, rotation and placement with great accuracy.

In another embodiment of the present invention, a player's physiological range through biosensors is predetermined and customized. For example, a player's set-point range of temperature when resting is 97° F. (36.1° C.) and when active 99° F. (37.2° C.). Another example, a player's resting heart rate is 60 beats per minute and his optimal performance heart rate is 134 beats per minute. The SGT device could be programmed to alert coaches when one or more player's heart rate is too high and exceeds his or her optimal range.

In another embodiment of the present invention, data acquisition mode of the wearable sensors can be changed automatically based on the predetermined set points so as to better characterize emergency or unusual situations. For example, when an accelerometer in the helmet or mouth guard of a football player exceeds a specified threshold during play, alerts and faster data acquisition are automatically triggered. Data is then collected at a much faster speed in order to evaluate possible concussive impact where rotational acceleration and rotational velocity could be largely increased. The alert can activate other sensors or biosensors such as heart rate, respiration rate, blood pressure sensors, etc. to acquire data at faster acquisition modes as well.

In another embodiment of the present invention the wearable sports system alerts coaches when a player's performance is suboptimal due to dehydration, heat-shock, illness, lactic acid build-up in muscles, lack of energy due to diet, or others. The wearable sports system enables coaches and trainers the ability to compare performance with a player's physiological attributes and thus know when to give him more play time or remove him from a game.

As optimal performance is analyzed by historical data and varies from player to player and from time to time, in another embodiment of the present invention, the wearable sports system and SGT device database tracks and analyzes, compares and reports performance in any activity or sport as it relates to physiological measurements.

In yet another embodiment, the wearable sports system and SGT device analyzes not only individual comparative (physiological, environmental, performance, kinematics) but also a team composite of energy levels.

In another example, an athlete such as a mountain climber, marathon runner, safari hunter, et al. when injured might not be able to communicate to rescuers about their injuries and/or location. In such circumstances, according to another embodiment of the invention, tracking wearable devices and physiological analytics could work in unison and communicate the athletes' injury and health status and exact location. This could save lives and assist paramedics to prepare well for injuries of injured athletes.

In yet another embodiment of invention, SGT device offers a way for those talented athletes who may suffer from non-disabling diseases or injuries to participate in and perform well in team and professional sports. In one example, a basketball player suffers from a heart arrhythmia and takes medication for the disease and is in care of a cardiologist. The disease does not negatively impact the player's daily life. However, the player is unable to play on the school team due to the heart condition. The SGT device measures the player's heart function, blood oxygen levels, and even blood medication levels to alert the coach when rest is needed and thus when the player needs to be replaced for short periods of time or to change roles on the team in real time to avoid precipitation of symptoms and harm. In a similar example, a football player has a leg injury. After a period of recovery, the player's muscles are still weak, resulting in changes to the player's gait when the muscles become fatigued. The SGT device assists the coach in determining when to remove the player by determining whether the player's gait is proper (e.g. by comparing the detected gait to a predetermined baseline for the player) or whether the player is susceptible to fall, allowing the player to rest and get medical treatment if needed. This could prevent further injuries without hindering the player's and team's success.

The present invention can be used in many such situations for several different sports in assisting athletes, coaches, and physicians to participate in sports and perform to best of their capacities without compromising their health.

In general, according to one aspect, the invention features a system comprising a device configured to be inserted or attached to an animal or human comprising a smart sensor receptacle for a sensor, the device further comprising one or more wearable sensors contained within or upon the receptacle, and at least one interface with a network configured to utilize the information obtained from the one or more sensors or from one or more platforms.

In embodiments, one or more functions of the device is selected from the group consisting of providing sports function, health analytics, diagnostic analytics, performance analytics; integration of wearable sensors, health-devices, sports and performance sensors on inanimate objects and sports equipment; sports gear, clothing, stadium, ballpark, park; gym, gymnasium, arena, dome, bowl, circus, coliseum, colosseum; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, historical analysis, time stamped data, reporting and feedback, positioning, the integrated video can be synced with all wearables and other biosensors in order to produce computer-generated precise movement and greater precision or analytics.

The system further comprises a database compilation of one or more players' performance entries, one or more players' physiological attributes, one or more players' kinematics. The system is further configured to analyze individual or team sports performance as it relates to various body components and sensors and provided with full connectivity and full server access. Additionally, the system is further configured to provide an alerting signal when outside a predetermined set point and to operate as a training and coaching network.

The system comprises physical tracking and precision field logistic software, a digital transactional communication interface and controls, navigation and operational guidelines configured to facilitate performance, software configured to provide athletic analysis, logistics, specialty location XYZ modules, date entry timestamp and input.

Additionally, the system is further configured to be customizable by a user in a sports practice or game mode to facilitate performance and optimal player health.

The system further comprises a historical database of the animal or human as to one or more characteristics from which comparisons or analyses are to be made, and means to optimize the play time of an individual player during a game or training.

The system includes smart data compiler software configured to data stream information for use by the user to evaluate one or more players' performances when playing in a sport or requiring an athletic performance.

The network is configured to carry out a functionality selected from the group consisting of signaling bi-directional transmissions to a secure server through one or more of WiFi, Bluetooth, GPS, NFC or other wireless means, temporarily storing information in the smart device, bi-directionally transmitting alert to pre-selected devices or pre-selected personnel. Additionally, the network is configured to analyze a composite input of a plurality of team or group members and interfaces with a mobile device or apparatus. The network interfacing with the mobile device provides sensor information or analysis to a user. Further, the network is capable of utilizing the information obtained from the one or more sensors comprises one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms. The network also includes one or more of measurements of performance, measurements of health, measurement of energy level, measurement of physiological attributes, information obtained from sensors, kinematics information, information obtained from cameras, information from sensors inserted or attached to body parts, information from instruments used to measure performance, information received from sensors attached to or associated with inanimate objects and sports equipment. The network also comprises means by which one or more sensors are activated by another sensor, device or remote controller and means for integrating one or more wearable sensors with sensors attached to or associated with inanimate objects or sports equipment.

In general, according to another aspect, the invention features a method of training comprising providing a virtual presentation of one or more athletes for visualization by one or more users.

In embodiments, the virtual presentation is configured to be three-dimensional profiles customizable by said user to facilitate performance. One or more data servers are provided for the user to virtually display three-dimensional profiles of one or more bodies or limbs for precise movement and analysis. A controller is further provided with the capacity to configure the database of one or more sensors and predetermined set points, scale, type of sport, athlete, individual energy alerting, team energy alerting, physiological computations, historical references, search engine and analytics. An analytical processing capability comprising motion and performance comparison is also provided. The virtual presentation of one or more athletes can comprise holographic images and patterns of synced simulations.

In general, according to another aspect, the invention features a method of training comprising utilizing a network of wearable sensors to guide a player or teams.

In embodiments, the network is configured to activate said wearable sensors to define said player's or teams' motions and/or to correct said player's motor skills and make adjustments to optimize said player's or teams' performance. Vibrations are utilized on a player's wearable devices to perform directional guidance, and artificial intelligence is utilized to determine an efficient motion for a player. The player's motion is corrected and/or adjusted through the wearable devices.

In general, according to another aspect, the invention features a method of training comprising utilizing robotics in a training or game to film, track or guide a player through the wearable devices.

In general, according to another aspect, the invention features a customizable tool kit or platform for building a wearable sports system to provide information, analysis or alerts for an animal, animals, human or humans, comprising a kit or platform of customizable components to meet the needs of a developer, consumer or user of the system, the components comprising at least one sensor inserted or attached to the animal, animals, human or humans, at least one receptacle configured to contain or receive the sensor, and at least one network unit configured to receive information, analysis or alerts from or transmit information, analysis or alerts to the at least one sensor and analyze, transmit, or both, the information, analysis or alerts obtained or received, wherein components for selecting the sensor receptacles, the sensors, and the network units are made available to the developer, consumer or user to construct or have constructed a wearable sports system configured to obtain or transmit information, analysis or alerts customized to meet the specific needs of the developer, consumer or user.

In embodiments, a preselected set of kit or platform components is provided in the kit or platform together with instructions for building the desired system. The system is designed for a sports function, health analytics, diagnostic analytics, performance analytics; integration of body sensors, health-devices, nano-particles, sports and performance sensors on inanimate objects and sports equipment; sports gear, clothing, stadium, ballpark, park; gym, gymnasium, arena, dome, bowl, circus, coliseum, colosseum; customizable developers' tool kit for biosensors, sensors, performance, medical analytics, oral and systemic diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics. The tool kit or platform further comprises a software control system configured to authenticate, analyze and gather data to guide, enhance performance. The tool kit or platform further comprises a software control system configured to provide one or more of the functions of tagging, tracking, logging data regarding smart sports equipment, smart sensor wearables as it relates to sports movement. The toolkit or platform further comprises a software control system configured to provide one or more of the functions of facilitating secure communication, adjusting motor skills, permeating smart particles and materials, entering secure data points and data sets which assist in coaching, training and athletic performance.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram depicting a sports analytics system according to one embodiment of the present invention;

FIG. 2 is a schematic diagram of the sports analytics system according to one configuration in which an external wearable device provides network connectivity between other devices of the sports analytics system;

FIG. 3 is a schematic diagram of the sports analytics system according to another configuration in which a user device provides the network connectivity;

FIG. 4 is a schematic diagram of the sports analytics system according to another configuration in which an autonomous mobile device provides the network connectivity;

FIG. 5 is a schematic diagram of an exemplary external wearable device;

FIG. 6 is a schematic diagram of an exemplary internal device;

FIG. 7 is a schematic diagram of an exemplary user device;

FIG. 8 is a schematic diagram of an exemplary autonomous mobile device;

FIG. 9 is a schematic diagram illustrating an exemplary sports analytics database;

FIG. 10 is a diagram illustrating an example of how the sports analytics system determines player performance based on sensor data;

FIG. 11 is a block diagram showing various exemplary registration packages for the sports analytics system;

FIG. 12 is a block diagram illustrating an example of an analytics and reporting system for an individual player;

FIG. 13 is a diagram illustrating how the sports analytics system generates sensor data based on internal devices such as sensors in oral cavities of the players;

FIG. 14 is a diagram illustrating how the sports analytics system generates sensor data based on internal devices such as smart mouth guards;

FIG. 15 is a diagram illustrating how the sports analytics system generates guidance information;

FIG. 16 is a diagram illustrating how the sports analytics system integrates external environmental factors;

FIG. 17 is a diagram illustrating how the sports analytics system analyzes physiological measurements in relation to performance;

FIG. 18 is an illustration of exemplary sensor set points, sensor data collection and alert and report generation;

FIG. 19 is an illustration of exemplary graphical representations of data collection generated by the sports analytics system;

FIG. 20 is a diagram illustrating how the sports analytics system analyzes kinematic factors to maximize performance;

FIG. 21 is an illustration of an example of how the sports analytics system analyzes kinematic factors to maximize performance;

FIG. 22 is a diagram illustrating how the sports analytics system functions as a fully integrated diagnostic and performance measurement system;

FIG. 23 is a diagram illustrating how the sports analytics system analyzes sensor data generated for an animal player such as a race horse; and

FIG. 24 is an illustration of different examples of external wearable devices and internal devices of the sports analytics system.

### DETAILED DESCRIPTION OF THE INVENTION

The foregoing summary, as well as the following detailed description of certain embodiments will be better understood when read in conjunction with the appended drawings. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein, the term “smart” means a device or object that performs one or more functions of a computer or information system, such as data storage, calculation, Internet access and information transmission.

As used herein the terms “insertable”, “implantable”, “imbeddable”, “embeddable”, “temporarily insertable” “permanently insertable”, “temporarily implantable”, “permanently implantable” , “temporarily imbeddable”, “permanently imbeddable”, “temporarily embeddable” and “permanently embeddable” refer to means of securely inserting and attaching in or to, or fastening a device, such as being adhered to, cemented, affixed or otherwise securely attached to a surface or object.

As used herein, the term “receptacle” refers to a device or container that receives, retains, has within, or holds something.

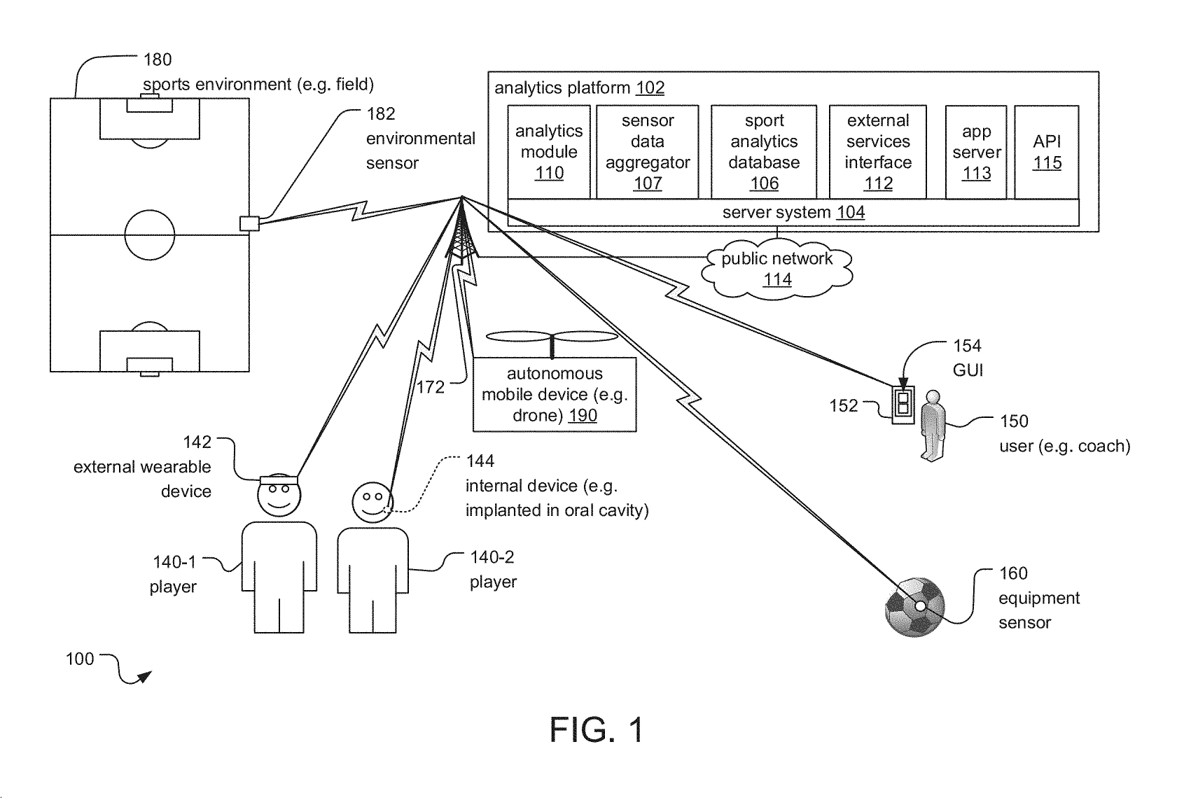

FIG. 1 is a schematic diagram depicting a sports analytics system 100 according to one embodiment of the present invention.

In general, the sports analytics system 100 aids players 140 and other users 150 such as coaches in improving performance in sports, including all land sports, ball sports, and water sports. The sports are played within a sports environment 180 such as a field, stadium, track, or pool, among other examples and can include team sports and individual sports. The players 140 are human or animal athletes which can be performing at any level, including professional, Olympic, college and high school sports.

The sports analytics system 100 includes an analytics platform 102, external wearable devices 142, internal devices 144, environmental sensors 182, equipment sensors 160, user devices 152, and autonomous mobile devices 190.

In general, the analytics platform 102 aids the players 140 and users 150 (e.g. coaches) by aggregating sensor data from the external wearable devices 142, internal devices 144, environmental sensors 182, equipment sensors 160, and autonomous mobile devices 190, analyzing the sensor data and other information, and providing information to the players 140 and the users 150. The sports analytics system 100 can be set up for use with an individual to obtain information from the individual and transmit information, or analysis derived from the information, directly or indirectly to a network or analytics platform 102.

The analytics platform 102 is capable of utilizing the information obtained from the one or more sensors and having functions including, but not limited to, data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

The analytics platform 102 also conducts measurements of performance, measurements of health, measurement of energy level, measurement of physiological attributes, information obtained from sensors, kinematics information, information obtained from cameras, information from sensors inserted or attached to body parts, information from instruments used to measure performance, information received from sensors attached to or associated with inanimate objects and sports equipment.

The external wearable devices 142 are configured to be worn by or attached to the players 140, while the internal devices 144 are configured to be inserted or implanted in the bodies of the players 140. The external wearable devices 142, internal devices 144 comprise smart sensor receptacles for sensors, one or more sensors contained within or upon the receptacle, and at least one interface with a network configured to utilize the information obtained from the one or more sensors or from one or more platforms, such as the analytics platform 102. The external wearable devices 142 might also include feedback mechanisms for providing information and/or to the players 140 such as vibrations, lights, or sounds, among other examples.

The equipment sensors 160 are embedded in or attached to sports equipment used by the players 140 such as balls, gloves, bats, hockey sticks, or golf clubs, among other examples.

The environmental sensors 182 are embedded in or attached to features of the sports environment 180. In one embodiment, the environmental sensors 182 generate sensor data indicating ambient temperature, humidity, altitude, wind speed and/or direction, barometric pressure, and air quality, among other examples.

The mobile computing device 152 presents information such as performance information for the players 140 to the coach 150 via a graphical user interface (GUI) 154. The mobile computing device 152 might also provide network connectivity between the analytics platform and the other devices of the sports analytics system 100 by, for example, relaying sensor data to and/or alerts from the analytics platform. In the illustrated example, the mobile computing device 152 is a smartphone device. Alternatively, the mobile computing device 152 could be a laptop computer, tablet computer, phablet computer (i.e., a mobile device that is typically larger than a smart phone, but smaller than a tablet), or smart watch, among other examples.

The autonomous mobile device 190 is an autonomous unmanned aerial vehicle or drone configured to automatically move through or around the sports environment 180, for example, by hovering over the playing field. The autonomous mobile device 190 includes network connectivity for communicating, for example, with the external wearable devices 142 and the analytics platform 102. The autonomous mobile device 190 further includes sensors for generating sensor data such as image data depicting the players 140 in motion during a game or practice.

The external wearable devices 142, internal devices 144, equipment sensors 160, environmental sensors 182, autonomous mobile device 190 and the user devices 152 communicate with the analytics platform 102 via a leased data connection, private network and/or public network 114, such as the internet. In the illustrated example, the devices connect to the public network 114 via wireless communication links to a cellular radio tower 172 of a mobile broadband or cellular network or public and/or private wired data networks such as an enterprise network, Wi-Max, or Wi-Fi network, for example. In practice, some devices of the sports analytics network 100 might provide network connectivity to the others, relaying sensor data from one or more devices to the analytics platform 100 for example. Additionally, some of the devices may be activated (via the network connection) by another sensor, device or remote controller.

Any of the external wearable devices 142, internal devices 144, equipment sensors 160, environmental sensors 182, autonomous mobile device 190 and the user devices 152 can further communicate via Radio Frequency Identification (RFID), near field communication, micro- and nano-communication protocols, for example, in order to send or receive the sensor data or other information such as identification information. Active and/or passive, and/or a combination of RFIDs use electromagnetic signals to uniquely distinguish and identify a mobile “TAG” device or stationary “TAG” device. The active RFID identification system tag has its own power source, enabling the unit to broadcast an identifying signal. This extends the range of the tags and capability of communicating advanced data, such as location and other pertinent information, and broadcasts an identifying signal. Passive RFID tags are not powered and rely on active signals from location transmitters for their response. RSSI (Received Signal Strength Indication) is an algorithm that determines the location of an active tag by measuring the power of the radio signals. TDOA (Time Difference of Arrival) is an algorithm that determines the location of active tags by measuring the power of radio signals in real-time. Some RSSI systems have choke-point capabilities that provide an instantaneous notice that a tag has passed a certain point. The various wearable devices 142 which communicate with one or more wireless devices, networks 102, drones 190, and subsystems (WiFi, satellites, cellular, etc.) which interface and communicate with the coach 150 or player 140.

The analytics platform 102 is typically implemented as a cloud system. It can be run on a proprietary cloud system or implemented on one of the popular cloud systems operated by vendors such as Alphabet Inc., Amazon, Inc. (AWS), or Microsoft Corporation.

As a result, the analytics platform 102 typically operates on a server system 104. In some cases, this server system 104 is one or more dedicated servers. In other examples, they are virtual servers.

The server system 104 executes a number of separate modules, including an analytics module 110, sensor data aggregator 107 and app server 113. Each of these modules is associated with separate tasks. In some cases, these modules are discrete modules or they are combined with other modules into a unified code base. They can be running on the same server or different servers, virtualized server system or a distributed computing system.

The sensor data aggregator 107 receives the sensor data generated by the external wearable devices 142, internal devices 144, environmental sensors 182, equipment sensors 160, and autonomous mobile devices 190 and stores the sensor data to a sports analytics database 106, which stores information about the teams and players 140.

The analytics module 110 analyzes information from the sports analytics database 106 such as sensor data and other information about the teams and players 140 and generates, for example, feedback and/or guidance information regarding a physiological characteristic of a current activity he is engaged in, such as running, jogging, walking, or a physical characteristic involved with playing a sport, and/or alerts, which the analytics module 110 then pushes to the user devices 152 of the coaches 150 and to the external wearable devices 142.

The information generated by the analytics module 110 might pertain to sports functionality, health analytics, diagnostic analytics, performance analytics, and integration of multiple different sensors such as health-devices, equipment sensors 160 on inanimate objects and on sports equipment and gear, environmental sensors 182 attached to or embedded within features of a sports environment 180 such as stadiums, ballparks, parks, gyms, arenas, domes, bowls, circuses, and coliseums. The analytics module 110 also provides a customizable developers' tool kit for sensors, including biosensors, performance, medical analytics, oral and systemic body diagnosis; integrated, pre-integrated and post-integrated platforms; analysis of any type of medium, secure bidirectional media, multiple media, video, audio, 3D, printing, reporting, analytics, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, historical analysis, time stamped data, reporting and feedback, positioning, the integrated video can be synced with all wearables and other biosensors in order to produce computer-generated precise movement and greater precision or analytics.

In one example, the analytics module 110 might be configured to analyze individual or team sports performance as it relates to the various body components such as external wearable devices 142 and internal devices 144 and other sensors such as the equipment sensors 160 and the environmental sensors 182.

In another example, the analytics module 110 includes tracking and precision field logistic software, based on sensor data from one or more sensors for temperature or acceleration.

In another example, the analytics module 110 includes a digital transactional communication interface and controls and generates navigation and operational guidelines configured to facilitate performance, and provides alerting signals when sensor data and biometrics indicate a player 140 or, collectively, a team, fall outside a pre-set range for biometric information.

The analytics module 110 can include software configured to provide athletic analysis, logistics, specialty location XYZ modules, and date entry timestamp and input.

Additionally, the analytics module 110 can include smart data compiler software configured to data stream information for use by the user 150 to evaluate one or more player's 140 performances when playing in a sport or requiring an athletic performance.

In yet another example, the analytics module 110 generates a virtual presentation of one or more players 140 for visualization by one or more users 150 including the players 140 themselves. These presentations may be configured to be three-dimensional profiles customizable by one or more users 150 to facilitate performance. For example, the analytics module 110 might generate a visualization including three-dimensional profiles of one or more bodies or limbs for precise movement and analysis. The presentations may further comprise holographic images and patterns of synced simulations through vibrations (e.g. of the external wearable devices 142) or multimedia for guidance and training.

The analytics module 110 further includes an analytical processing capability comprising motion and performance comparison.

In another example, the analytics module 110 generates training information based on the sensor data. The training information includes information to guide and train individuals or teams. The analytics module 110 determines the players' 140 motion based on the sensor data and corrects the player's 140 motor skills and offers adjustments to optimize the players' or team's performance.

In yet another example, the analytics module 110 utilizes artificial intelligence to determine precise movements for a player is provided. The artificial intelligence can be customized to correct or adjust a player's motion through the wearables.

In one embodiment, the analytics module 110 analyzes sensor data indicating biometric measurements pertaining to the players 140 (e.g. SpO2, pulse, temperature, blood pressure, hydration) and generates feedback and guidance information based on a comparison of the biometric measurements with sensor data indicating one or more additional aspects of the performance, health, technique and/or environment of the players 140 including: sensor data (e.g. generated by environmental sensors 182) indicating ambient temperature, humidity, altitude, wind, barometric pressure, air quality; sensor data (e.g. generated by external wearable devices 142 with gyroscopes, microtometers, pressure sensors, force sensors, and other redundant body sensors) indicating precise movement information such as musculoskeletal information, motor change of posture, and muscle activity; and sensor data (e.g. generated by internal devices 144 such as a smart mouth guard with sensors that detect conditions of the saliva of the players 140) indicating chemical and/or biological conditions of the players 140 changes in lactic acid, nano particles, graphene pedals and PH levels. The analytics module 110 might analyze the sensor data indicating the precise movement information against movement filters indicating different types of movement and generate feedback and/or guidance information for improving the movement and increasing accuracy of the players 140. The analytics module 110 might gather performance information for players 140 over a period of time (e.g. intervals, scoring, weights, speed, and/or distance achieved) and compare the performance information with sensor data indicating the chemical and/or biological conditions of the players 140 and generate individualized optimal bio-metric information for each player 140 such as optimal ranges for best performance for various biometrics such as pulse or SpO2, among other examples.

In one example, the analytics module 110 determines, based on comparing the performance information and the biometric information that the optimal pulse range for a player 140 is 133-138 BPM and establishes a danger zone for the player 140 of 150+ BPM. The analytics module 110 might then send feedback information to the player 140 and/or users 150 such as the coach via the user devices 152, the feedback information indicating that the player 140 should take actions to increase or decrease their pulse in order to get to or remain in the optimal range. When that player's 140 pulse is greater than 150 BPM, the analytics module 110 might send an alert to the user devices 152 and/or the external wearable devices 142 indicating that the pulse is in the danger zone, and the player 140 should take action to decrease the pulse and/or the coach 150 should remove the player 140 from the game.

In a similar example, the analytics module 110 defines an optimal SpO2 range for a particular player 140 as 99.5-100 in response to determining that the player 140 averages scoring thirty points per game while in that range but only twenty-two points per game while below 99.5 based on the sensor data and detected/received performance information (e.g. score information input by the coach 150 via the GUI 154 of the user device 152 or detected via video analytics). The analytics module 110 might then receive and monitor the sensor data indicating the SpO2 of the player 140 in real time (e.g. during a game or practice) and generate feedback information based on how the current SpO2 compares to the optimal range. As before, the analytics module 110 sends the feedback information to the external wearable devices 142 and/or the user devices 152 to be presented to the players 140 and the users 150.

In a similar example, the player 140 might be a race horse, and the analytics module 110 determines based on the sensor data received from the external wearable devices 142 and the internal devices 144 of the race horse that the optimal pulse for that race horse is 180-190 BPM in response to determining that lap times drop by 0.2% when the sensor data for the race horse indicates that the pulse is 190+ BPM. The analytics module might also define a pulse of 250+ BPM as the danger zone for the race horse and generate and send alerts to the user devices 152 based on the real time pulse of the race horses.

In each case, the external wearable devices 142 present the feedback information and/or alerts via the feedback elements 412, for example, by displaying the information, vibrating, and/or playing a message through speakers. Similarly, the user devices 152 might display the feedback information and/or alerts via the GUI 154.

In another embodiment, the autonomous mobile device 190 (e.g. a drone) captures image data depicting a player's 140 such as a race horse's movements while running in a race from multiple different angles. The analytics module 110 receives the image data and generates precise movement information based on the image data. The analytics module 110 might then generate a virtual model of the movement to be displayed by the user devices 152, or it might generate feedback information, for example, indicating how the movement might be improved or whether any problems were detected based on comparing the precise movement information to one or more movement filters.

In the preferred embodiment, the sensor data, feedback information, guidance information, alerts, and/or any other information exchanged between the devices and the analytics platform 102 are encrypted to prevent third-party access of the information. In one example, the encryption includes pre-encrypting the information before sending it as well as bonding/link-level encryption at each node of the network between the origin and destination of the information.

The app server 113 communicates with the user devices 152 by, for example, processing the information generated by the analytics module into a visual format (e.g. charts, graphs, diagrams) and pushing the information to the user device 152. The app server 113 also receives information from the user devices 152 input by the users 150 via the GUI 154 and, in different examples, stores the information in the sports analytics database 106 or sends the information to the analytics module 110 to be processed.

The analytics platform 102 also includes an external services interface 112, which operates as the interface between the analytics platform 102 and services operated independently of the analytics platform 102 such as those providing sports statistics information, or health and fitness tracking information generated by devices and services outside of the sports analytics system 100, among other examples. The external services interface 112 puts the information retrieved from the external services into a format that can be consumed by the analytics module 110 and/or stored in the sports analytics database 106.

In one example, the smart sensor receptacle is a head band and the smart sensor receptacle is configured with WiFi connectivity. In another example, the smart sensor receptacle is an arm band with full connectivity, the system further includes full server access and is configured for an analytical processing capability. In another example, the smart sensor receptacle is a full or partial retainer, the system further includes a smart mouth guard accessory, the one or more sensors includes sensors for temperature or oxygen levels, and the system is further configured with WiFi connectivity and is configured to provide an alerting signal when the temperature or oxygen levels are outside a pre-set range. In another example, the smart sensor receptacle is an ear bud, the system is provided with full connectivity and full server access and is configured for an analytical processing capability comprising performance analysis.

In one embodiment, the sports analytics system 100 is a customizable tool kit for building a system to provide the information, analysis or alerts as previously described. The kit comprises a customizable set of components such as external wearable devices 142, internal devices 144, equipment sensors 160, environmental sensors 182 and/or autonomous mobile devices 190 to meet the needs of a developer, consumer or user 150 of the system. In one example, the analytics platform 102 is configured to obtain or transmit information, analysis or alerts customized to meet the specific needs of the developer, consumer or user via an API 115 executing on the server system 104.

In an embodiment of the system, the tool kit or platform of the wearable sports system comes in a variable grouping of preselected sets of kit or platform components or modules of components for constructing the wearable sports system using the kit or platform, and may come together with instructions for building the desired system. And yet further, in certain embodiments, at least one smart auxiliary component is present in the tool kit or platform.

The tool kit or platform as outlined above, e.g., can be designed for sports functions, health analytics, diagnostic analytics, performance analytics; integration of body sensors, health-devices, nano-particles, sports and performance sensors on inanimate objects and sports equipment; sports gear, clothing, stadium, ballpark, park; gym, gymnasium, arena, dome, bowl, circus, coliseum, colosseum; customizable developers' tool kit for biosensors, sensors, performance, medical analytics, oral and systemic diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

The tool kit or platform in another embodiment includes but is not limited to a software control system configured to authenticate, analyze and gather data to guide and/or enhance performance.

The tool kit or platform in another embodiment includes but is not limited to a software control system configured to provide one or more of the functions of tagging, tracking, and/or logging data regarding smart sports equipment and/or smart sensor wearables as it relates to sports movement.

The tool kit or platform in yet another embodiment includes but is not limited to a software control system configured to provide one or more of the functions of facilitating secure communication, adjusting motor skills, permeating smart particles and materials, entering secure data points and data sets which assist in coaching, training and athletic performance.

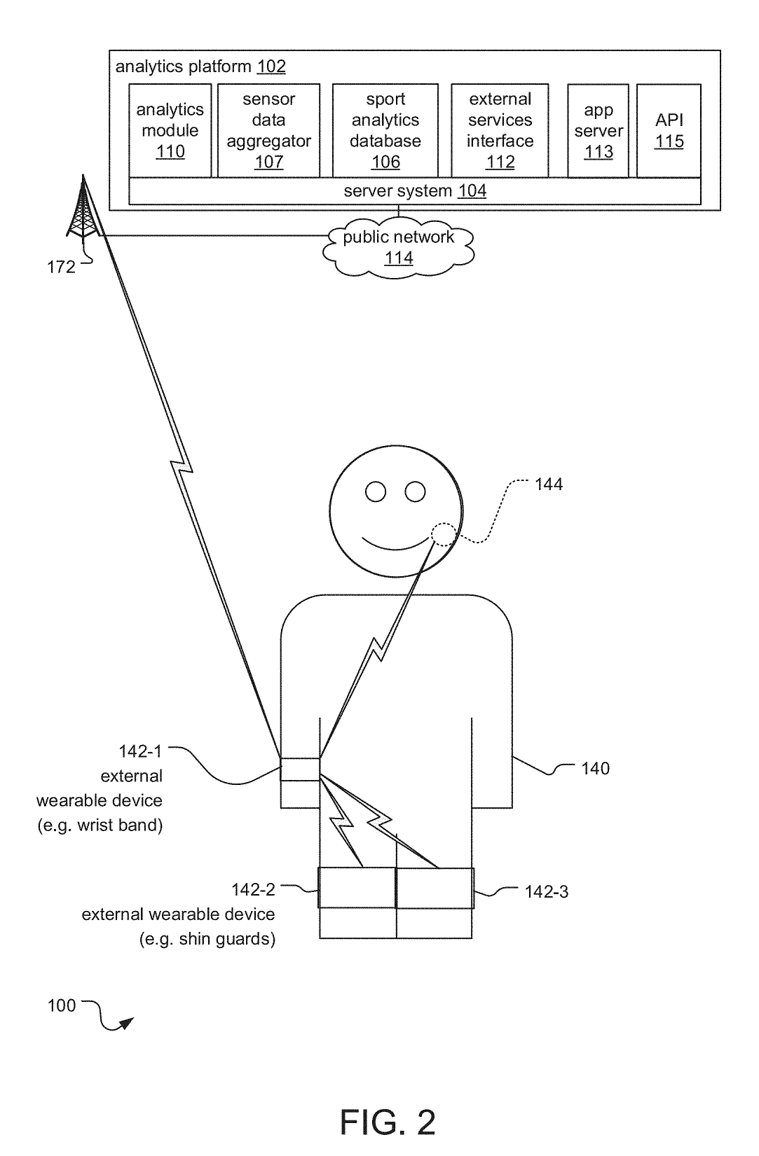

FIG. 2 is a schematic diagram of the sports analytics system 100 according to one configuration in which an external wearable device 142-1 provides network connectivity between other external wearable devices 142-2, 142-3, the internal device 144, and the analytics platform 102. In the illustrated example, the player 140 wears three external wearable devices 142, a wrist band 142-1, and two shin guards 142-2, 142-3. The player 140 also has an implanted internal device 144 in the player's 140 oral cavity. The wrist band 142-1 receives sensor data from the shin guards 142-2, 142-3 and the internal device 144 and relays the sensor data, along with any sensor data generated locally by the wrist band 142-1, to the analytics platform 102.

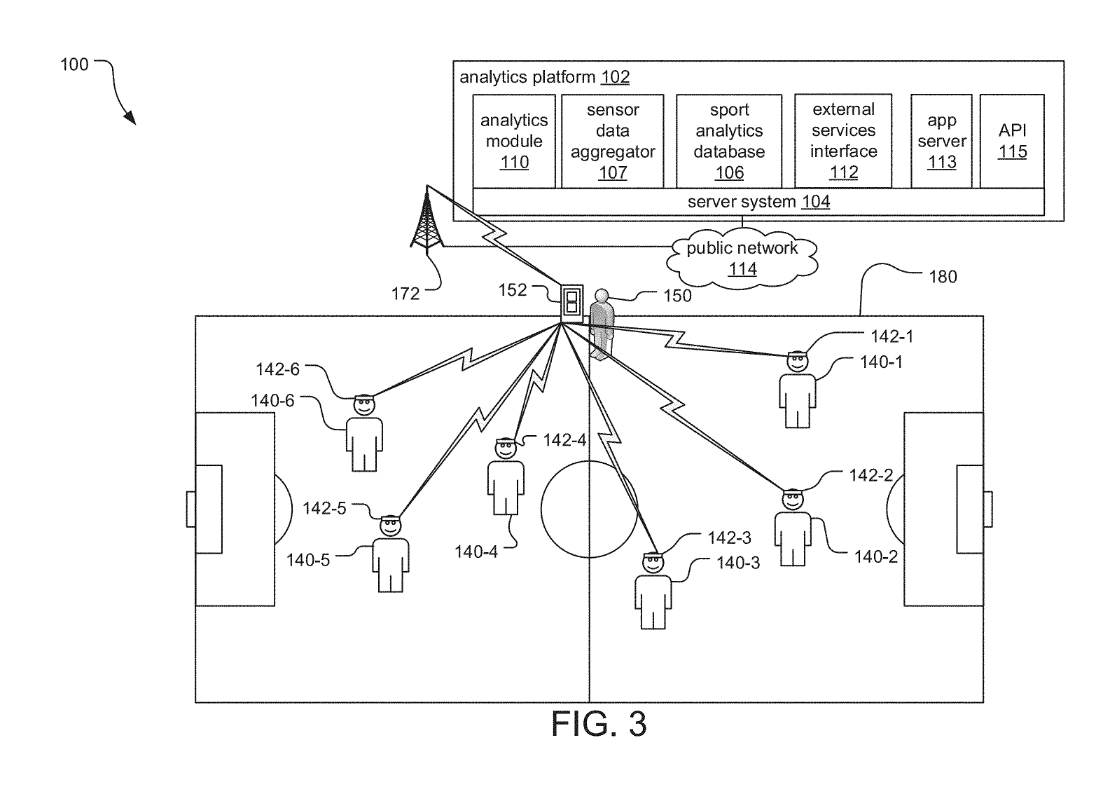

FIG. 3 is a schematic diagram of the sports analytics system 100 according to another configuration in which the user device 152 operated by the coach 150 provides network connectivity between the external wearable devices 142 of the players 140 and the analytics platform 102. In the illustrated example, six players 140-1, 140-2, 140-3, 140-4, 140-5, 140-6 are distributed across the field 180, each respectively wearing an external wearable device such as a head band 142-1, 142-2, 142-3, 142-4, 142-5, 142-6. The user device 152 receives sensor data from the head bands 142-1, 142-2, 142-3, 142-4, 142-5, 142-6 (e.g. sensor data generated locally by the head bands or sensor data received from other devices) and relays the sensor data to the analytics platform 102.

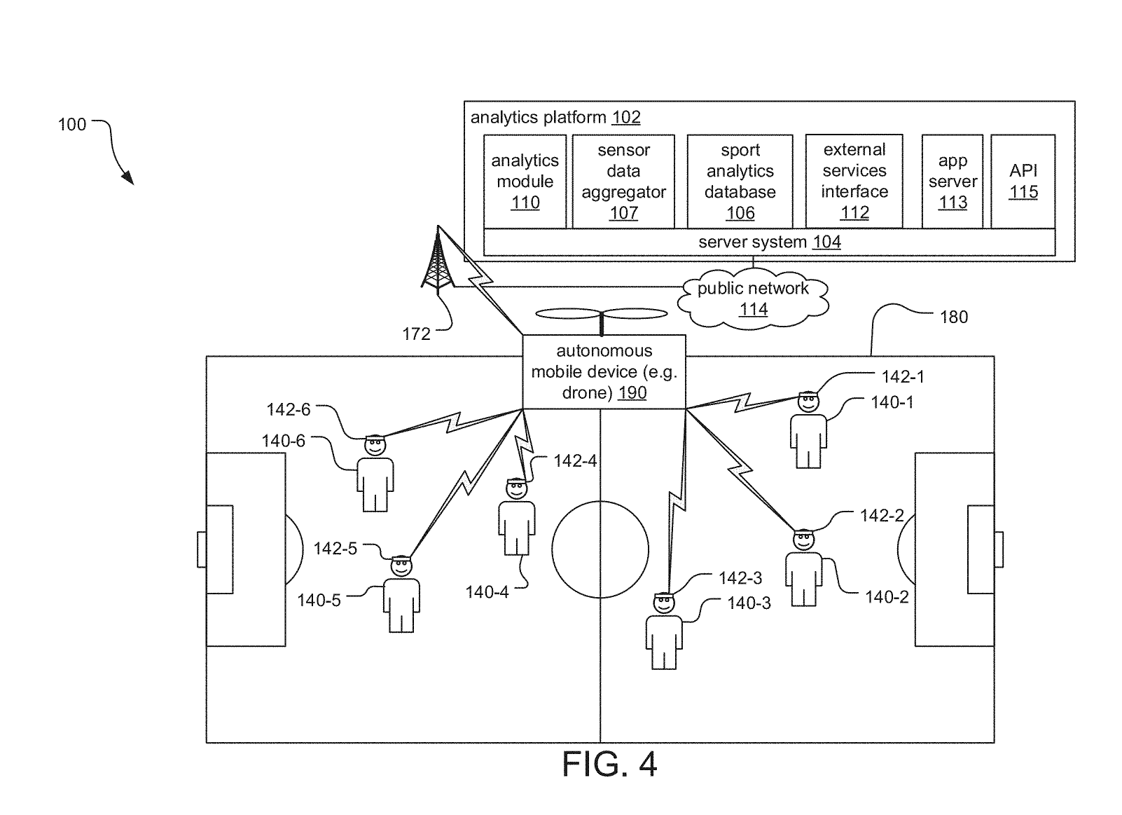

FIG. 4 is a schematic diagram of the sports analytics system 100 according to another configuration in which the autonomous mobile device 190 provides network connectivity between the external wearable devices 142 of the players 140 and the analytics platform 102. Similar to the example depicted in FIG. 3, the six players 140-1, 140-2, 140-3, 140-4, 140-5, 140-6 are distributed across the field 180, each respectively wearing head bands 142-1, 142-2, 142-3, 142-4, 142-5, 142-6. Now, however, the autonomous mobile device 190, which might be a drone hovering over the field 180, relays the sensor data from the head bands to the analytics platform 102. In this example, the autonomous mobile device 190 also generates position information for the players 140 based on the sensor data or wireless signals received from the external wearable devices 140 and sends alerts to the players 140 based on the position information.

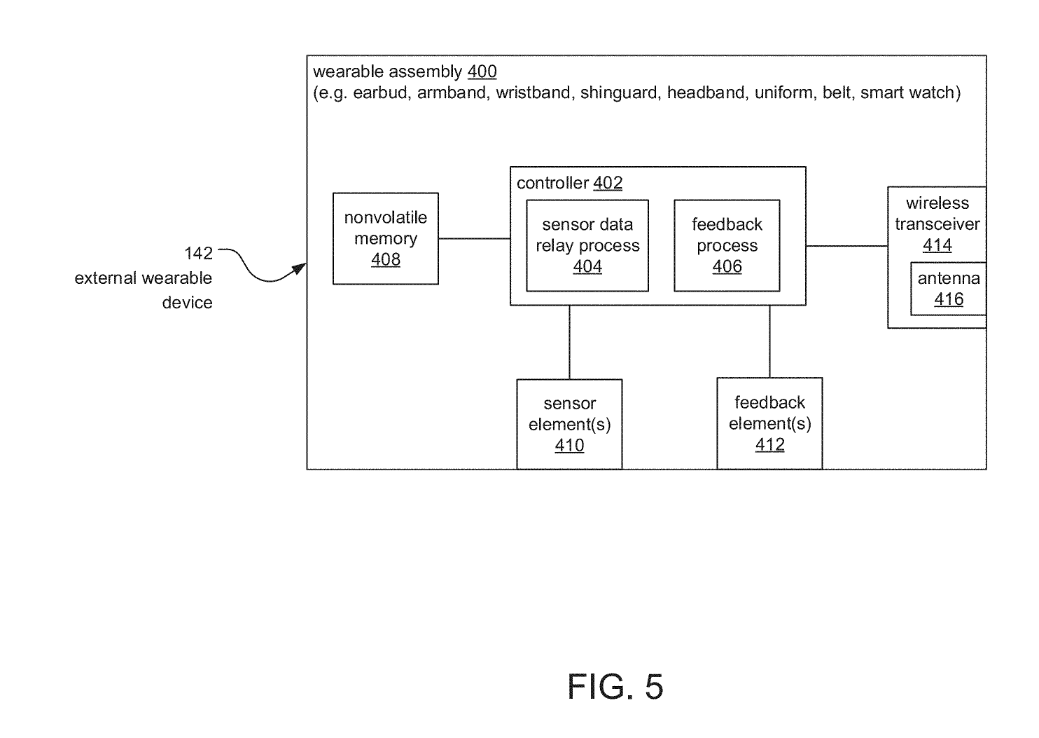

FIG. 5 is a schematic diagram of an exemplary external wearable device 140.

The external wearable device 140 includes a wearable assembly 400, a controller 402, nonvolatile memory 408, a wireless transceiver 414 and antenna 416, as well as one or more sensor elements 410 and feedback elements 412.

The wearable assembly 400 is a wearable object such as clothing, sports gear, arm bands, earbuds, wristbands, shin guards, head bands in which the other components of the external wearable device 142 are embedded or attached.

The wireless transceiver 414 and antenna 416 facilitate wirelessly sending and receiving bi-directional transmissions to a secure server such as the analytics platform 102 or other devices using wireless technologies such as WiFi, Bluetooth, GPS, NFC or other wireless means.

The controller 402 executes firmware instructions along with processes associated with managing the functionality of the sensor elements 410 and feedback elements 412. In particular, a sensor data relay process 404 and a feedback process 406 execute on the controller 402. The sensor data relay process 404 receives sensor data generated by other devices (such as internal devices 144 or other external wearable devices 142) and sends the sensor data to the analytics platform 102 via the wireless transceiver 414 and antenna 416. The feedback process 406 receives feedback information and/or alerts from the analytics platform 102 and, based on the feedback information and/or alerts, provides physical feedback to the players 140 via the feedback elements 412.

The sensor elements 410 are sensors or input mechanisms for generating sensor data and might include, e.g., sensors of blood pressure, core body temperature, heart rate or pulse, blood oxygen levels (e.g. SpO2), hydration levels, levels of a predetermined biologic, chemical or medication or their metabolites, sensors to measure a physical property, including one or more sensors which measure a physical property including or consisting of temperature, blood pressure, teeth pressure, ionic conductivity, airflow, images, optical density, alterations to the oral cavity, surrounding muscle tone, muscle weakness, heart rate, heart rhythms, respiration rate, accelerometer, accelerometer arrays, tri-axial accelerometers, gyroscopes, tri-axial gyroscopes, pressure sensors, magnetometers, goniometers, spectrophotometry, electromagnetic spectrum, gamma waves, X-ray waves, ultraviolet waves, visible waves, infrared waves, terahertz waves, microwaves, radio waves, electrical waves, sound waves, magnetic waves, ultrasonic waves, magnetic resonance, magnetic field, electro- or magnetic-encephalography, functional magnetic resonance imaging, optical topography, global positioning or tracking, accelerometer activity, gyroscopic activity, kinematic activity and radiation wave activity.

The feedback elements 412 are physical actuators and/or outputs for presenting information to the players 140, including, for example, vibration motors for creating a vibration sensation, light emitters, display indicators, or display screens, and/or speakers, among other examples.

The nonvolatile memory 408 stores sensor data for future transmission, for example, when network connectivity is not available.

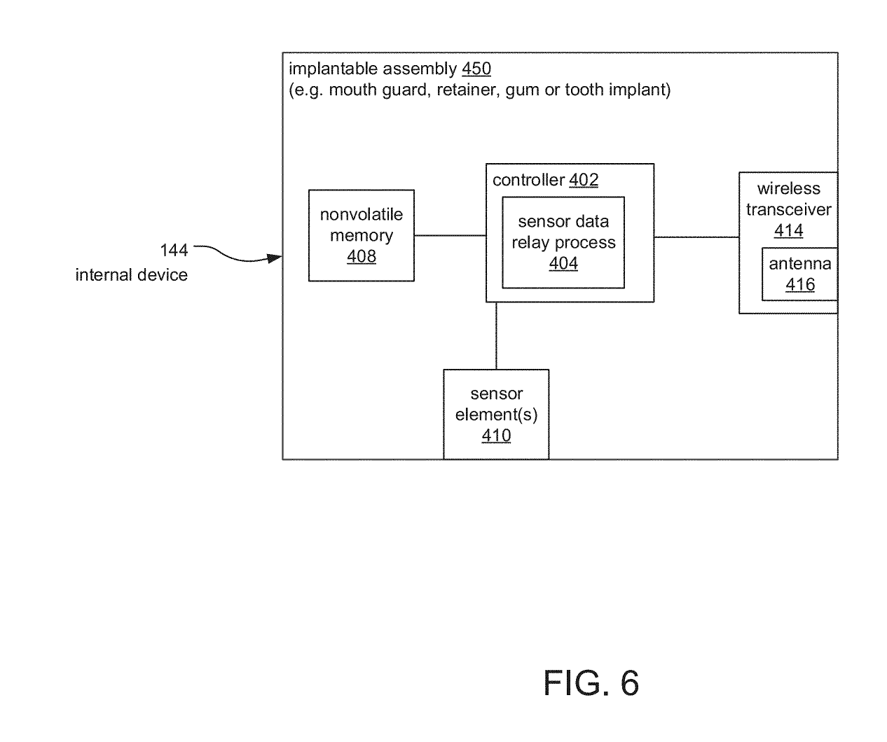

FIG. 6 is a schematic diagram of the internal device 144.

As with the external wearable device 142, the internal device 144 includes the controller 402 executing the sensor data relay process 404, nonvolatile memory 408, wireless transceiver 414 and antenna 416, and sensor elements 410. Now, however, these components are contained in an implantable assembly 450, which is an object that can be inserted or attached inside the player's 140 body. In different examples, the implantable assembly 450 can be a mouth guard, retainer, or oral implant attached to or near the teeth or gums.

In the illustrated example, the internal device 144 does not include the feedback elements 412.

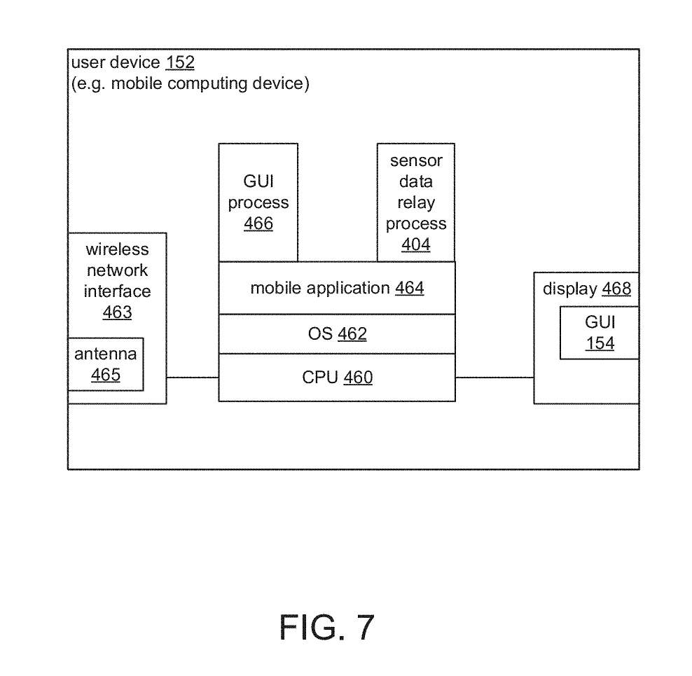

FIG. 7 is a schematic diagram of the user device 152 operated, for example, by the coach 150. The device, which might be a mobile computing device, includes a CPU 460, a touchscreen display 468, and the wireless transceiver 414 and antenna 416.

The CPU 460 executes firmware/operating system instructions and sends instructions and data to and receives data from the wireless network interface 463, the display 468 and other hardware components of the user device 152 (not illustrated). Executing on typically an operating system 462 of the CPU 460 is a mobile application 464 as well as drivers, for example, for directing the functionality of the wireless network interface 463 and/or display 468.

In general, the wireless network interface 463 sends and receives information between the user device 152 and the app server 113 of the analytics platform 102 via the antenna. The wireless network interface 463 also facilitates communication between the user device 152 and any other devices of the sports analytics network 100 such as the wearable external devices 142, internal devices 144, equipment sensors 160, environmental sensors 182 and autonomous mobile device 190.

The mobile application 464 includes a graphical user interface (GUI) process 466. In general, the GUI process 466 renders the GUI 154 on the touchscreen display 468. The GUI 154 includes a series of screens for displaying information and receiving input from the user 150, for example, by detecting contact between the user 150 and the touchscreen display 468 in certain regions of the touchscreen display 468. The GUI process 466 generates graphical elements (such as icons, virtual buttons, or menus) to be displayed via the GUI 154 and receives user input indicating selections of options represented by the graphical elements of the GUI 154.

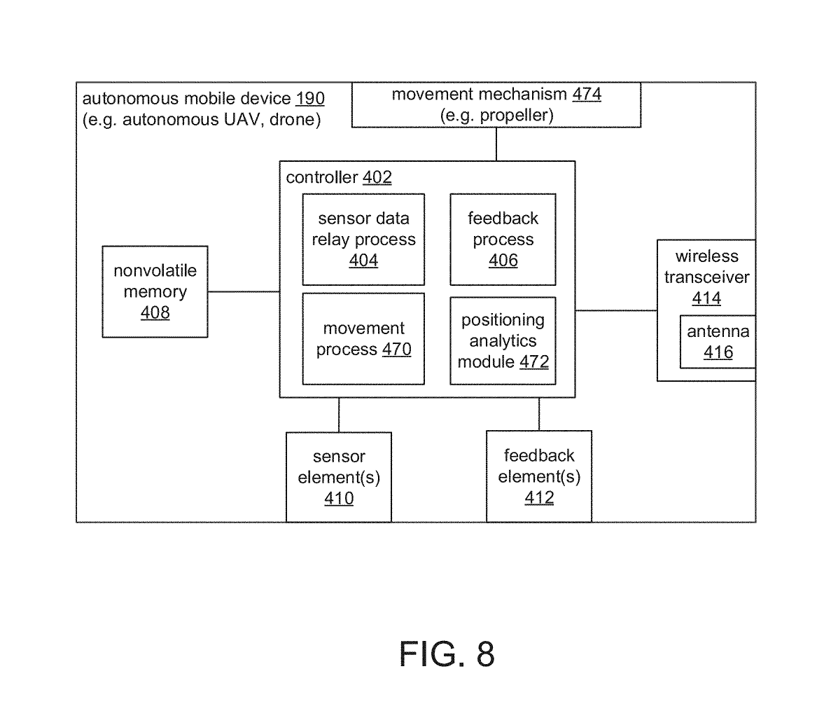

FIG. 8 is a schematic diagram of the autonomous mobile device 190. As with the wearable external device 142 and the internal device 144, the autonomous mobile device 190 includes the controller 402 executing the sensor data relay process 404 and the feedback process 406, the nonvolatile memory 408, the wireless transceiver 414 and antenna 416, the sensor elements 410 and the feedback elements 412.

In addition, however, the autonomous mobile device 190 includes a movement mechanism 474, which is a physical actuator enabling the autonomous mobile device 190 to move around the sports environment 180. In one example, the autonomous mobile device 190 is a drone hovering over the field, and the movement mechanism 474 is one or more propellers.

The movement mechanism 474 is controlled by a movement process 470 executing on the controller 402. The movement process 470 generates movement instructions based on sensor data generated by the sensor elements 410 and information and/or instructions from the analytics platform 102. The movement process 470 then sends the movement instructions to the movement mechanism 474, which causes the autonomous mobile device 190 to move according to the movement instructions.

The autonomous mobile device 190 also includes a positioning analytics module 472 executing on the controller 402. The positioning analytics module 472 generates position information for the players 140 based on sensor data received via the sensor elements, including, for example, image data captured by a camera and/or wireless signals emitted by the external wearable devices 142. The positioning analytics module 472 sends instructions to the feedback process 406 executing locally or to other devices such as the external wearable devices 142 to provide feedback and/or to alert players based on the position information. For example, to inform a first player 140 of the current position of a second player 140, the positioning analytics module 472 might send instructions to the feedback process 406 to emit a laser beam indicating the position of the second player, or to one of the external wearable devices 142 of the first player (such as a left or right armband) to vibrate to indicate the position of the second player 140.

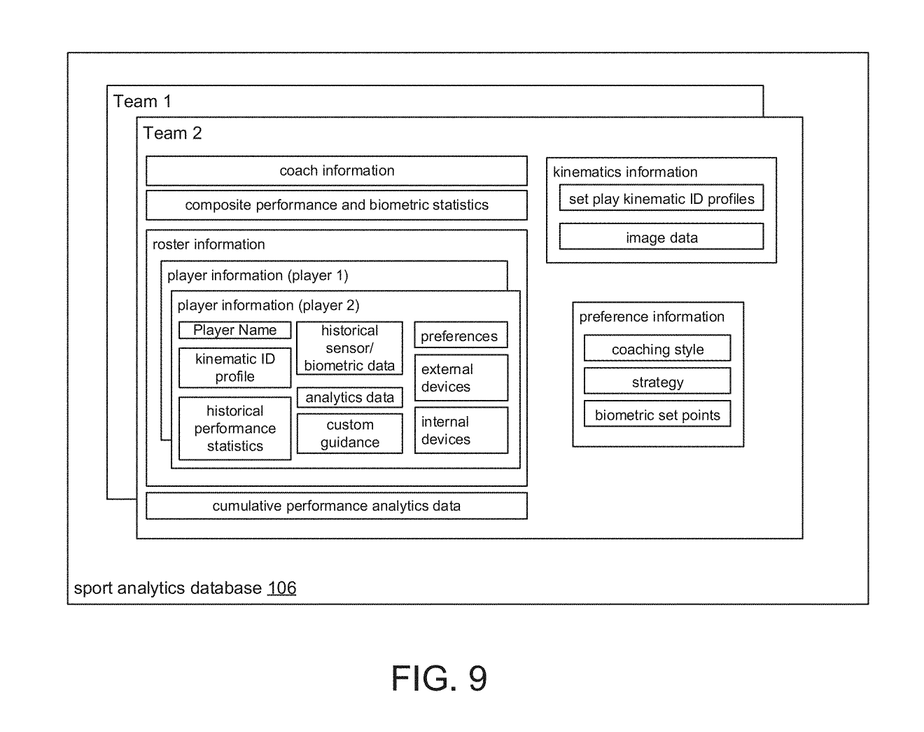

FIG. 9 is a schematic diagram illustrating an exemplary sports analytics database 106. The sports analytics database 106 stores team data as well as externally and internally integrated analytics (such as sensor data received via the sensor data aggregator 107 and/or other information received via the external services interface 112). In one embodiment, each specific team is a branch derived from the main database and has its own firewall protected storage database. Team and player information for a particular team is viewable by members of that team such as players 140 and other users 150 associated with the team such as coaches and/or trainers.

Generally, the sports analytics database 106 might include information about the human or animal players 140 as to one or more characteristics from which comparisons or analyses are configured to be made, or a database of animals or humans having a common characteristic to the animal or human on which the smart device is located and for which a predetermined comparison is configured to be made.

The database might also include a compilation of one or more players' biological or physiological attributes as they relate to one or more players' performances or one or more players' kinematics as they relate to one or more players' performances.

The database might further be configured to store sensor data from one or more sensors and predetermined set points, scale, types of sports, athletes, individual energy thresholds for generating alerts, team energy thresholds for generating alerts, physiological computations, historical references, search engine and analytics.

In the illustrated example, the sports analytics database 106 includes information about different teams and players 140.

Each team includes information about the coach, composite performance and biometric statistics (e.g. for all players 140 of the team), roster information including individual player profiles for each player 140, cumulative performance analytics data (e.g. collective energy level information based on composite biometrics and sensor data for all players 140), kinematics information including archived image data depicting the players 140 of the team (for example, during games and/or training sessions) and set plays associated with kinematic identification (ID) profiles for evaluating plays depicted by the image data against the intended plays, and preference information, which might include information about coaching style, strategy and/or biometric set points (for example, for determining whether alerts should be generated).

The individual player profiles include information such as player names and other identifying information such as uniform numbers and pictures or images depicting the player, a kinematic ID profile for identifying the players 140 based on analyzing the image data, historical performance statistics, which might be generated based on sensor data, input by users 150 via the user devices 152 and/or retrieved from external services via the external services interface 112, historical sensor/biometric data, analytics data (e.g. optimal biological standards and kinematic IDs) and custom guidance information generated by the analytics module 110, personalized preferences for the players 140, as well as identification information for all of the external wearable devices 142 and/or internal devices 144 associated with the particular player 140.

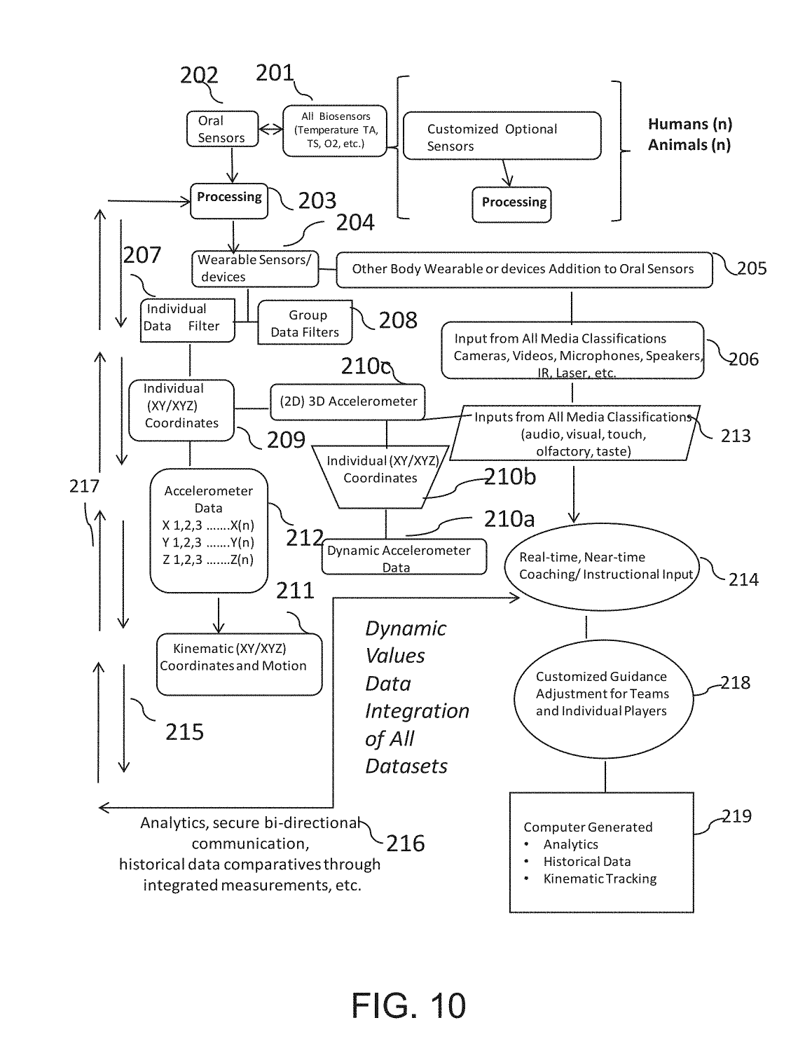

FIG. 10 is a diagram illustrating an example of how the sports analytics system 100 determines player 140 performance based on sensor data. The illustrated example shows a fully integrated performance measurement system including different types of biosensors (e.g. accelerometers, gyroscope), the selection and implementation of which could be standardized or customized and provided as a customizable tool kit for tracking humans and animals.

In 210c, 2D or 3D accelerometer models, which dynamically distinguish both an Individual Data filter 207, and Group Data filters 208, of 2D and 3D models, multiple visual sensors, for example, analyzing image data depicting a sports match to distinguish geometric and mathematical relationships between players, the equipment sensor 160 (e.g. smart basketball or other ball, smart hoop, smart baseball, smart bat, smart gloves, etc.), external wearable devices 142 worn by athletes and animals on any part of the body (e.g. head, upper-back, lower back, legs, knees, shoulder, elbow, hip, ankle, armpit, hand, glasses, contact lens, foot, toe).

Real-time or near-time reporting 214 and comprehensive database and historical data analysis and bi-directional communications 217 for authorized coaches and managers are also provided.

Customized guidance adjustment for teams and individual players is presented in 218.

In 203, advanced computer processing is indicated which can evaluate one or more variables originating from an individual (or animal), including, for example, 202 oral biosensor and 201 biosensor data such as TA, TS, O2, etc., 205 wearables worn on the body, 206 input from all media and other sources (temperature, accelerometer, gyroscope, inertia-sensor, tracking, sensors, camera, video, microphone, speakers, video, speakers, IR, thermal, sensors, positioning, laser, gyroscope, etc.), 213 input from all media, classifications (audio, visual, touch, olfactory, taste, etc.), and 210a dynamic accelerometer data 209 athletes position tracking (XY), indoor positioning (XYZ) and all other data sources. The integration and amalgamation of the aforementioned input data can comprehensively 209 integrate one player's data on a team or 208 multiple players' data on one or more teams in order to integrate the above with 209 positioning, movement and 211 kinematic relationships from multiple modes.

The resulting SGT processed data can utilize probabilistic data association and analytic deterministic data which could help lessen kinematic interference from multiple angles and positions as exemplified in 212. The SGT will provide coaches and managers, for example, integrated tools and greater accuracy as to both a player's physical health and energy, but as it relates to precise movements (210b).

The SGT device collectively provides the coach, trainer or manager 217 secure bi-directional communications, comparatives, historical analysis, time stamped data, reporting and feedback.

Individual “wearable” data can be used as part of a team composite calculated from a plurality of wearable “inter” and “intra” devices. Smart Inter-devices (SIRD) (e.g. internal devices 144), for purposes herein, are devices which can be implanted in the oral cavity, for example. Smart External Wearable Devices (SEWD) (e.g. external wearable devices 142) are defined herein as devices which can be inter-operationally worn on the body or near the body of the player 140. External Structures (ES) (e.g. environmental sensors 182) can be defined by any structure, such as, but not limited to, a playing field, stadium, racetrack, court, including any indoor or outdoor environment, which facilitates an athletic or organizational team. Smart Sports Equipment (“SSE”) (e.g. equipment sensors 160) is defined as any equipment needed to facilitate their respective sport and the sport's athlete; such as smart-balls, smart-hoops and smart-base-boards and any other device which facilitates their respective sport. Such sports equipment, e.g., smart-balls, can be tracked, their movements traced, mapped and integrated by means known to those skilled in this art.

In sports, 3D situations can be kinematically ambiguous, or at least very difficult from a tracking algorithm standpoint to be accurately established due to, for example, body parts being close together (e.g., an arm may be pressed against, and blend into another player's back, etc.) when videotaping a sports match or training session. High-definition videos can be constructed or reconstructed when a network of athletes is equipped with smart-wearables, thus helping solve movement ambiguities when integrated and synced with biosensors, wearables, and video. The integrated video can be synced with data produced by all wearables and other biosensors in order to produce computer-generated precise movement and greater precision and analytics as shown in 216 and 219.

To increase positive training (e.g., using vibrational, visional or auditory guidance through wearables and other smart accessories for individuals or, collectively, team guidance, and thereby make performance adjustments determined and set by a coach or staff) skills and greatly enhance performance. Players and coaches can use a variety of smart formats and cellular and wireless platforms to communicate with ear pieces and by other means.

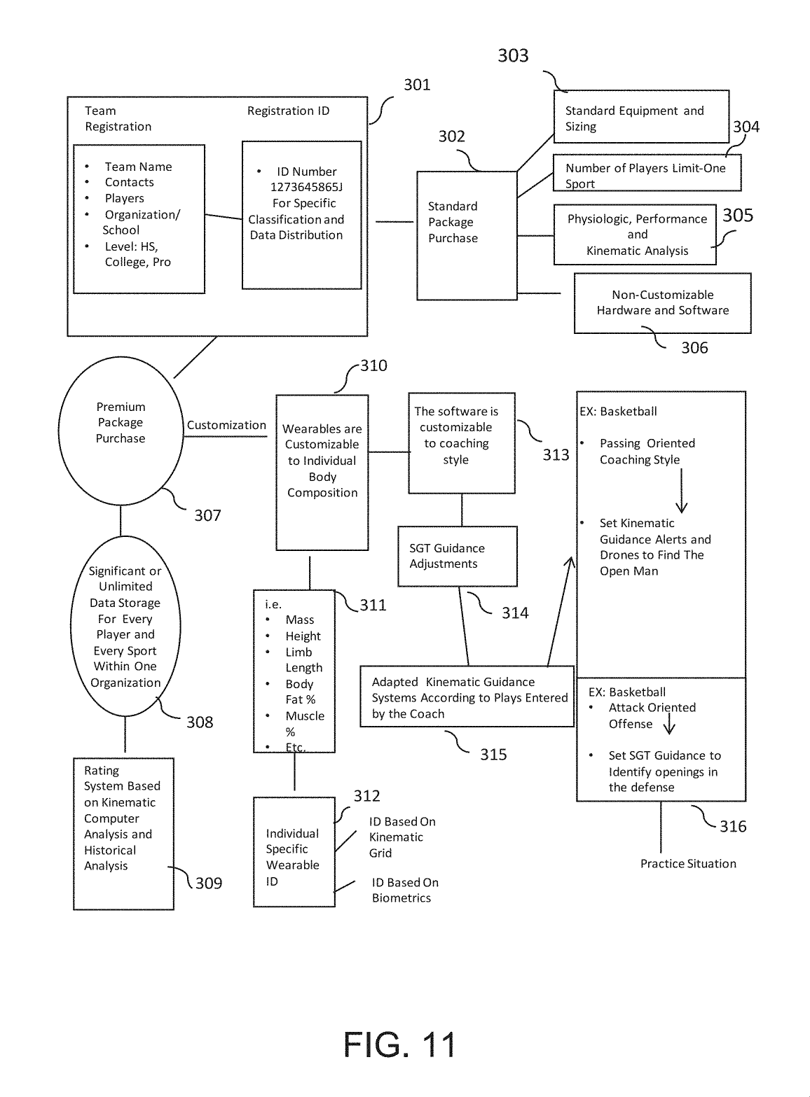

FIG. 11 is a block diagram showing various exemplary registration packages. When a team registers, it will be given a registration ID for specific classification and data distribution 301. Registration information consists of team name, contacts, players 140, organization/school and professional level etc. Standard package 302 is limited to one sport only and has a fixed number of players 304. The package provides standard equipment and sizing 303, non-customizable hardware and software 306, physiologic, performance and kinematic analysis 305, etc. Premium package offers significant or unlimited storage for every player and every sport within one organization 308. It also generates a composite rating system based on kinematic computer analysis and historical analysis 309 etc. Both hardware 310 and software 313 are customized. For example, wearables are customizable to individual body composition, i.e. mass, height, limb length, body fat % and muscle % etc. 311 Individual specific wearable ID 312 is given based on kinematic grid and/or physiologics. The software 313 is customizable to coaching style. SGT adjustments 314 are adapted kinematic guidance systems according to plays entered by the coach 315. For example, during basketball practice, a passing oriented coaching style can set kinematic guidance alerts and drones to find the open man while an attack oriented offense can set SGT guidance to identify openings in the defense 316.

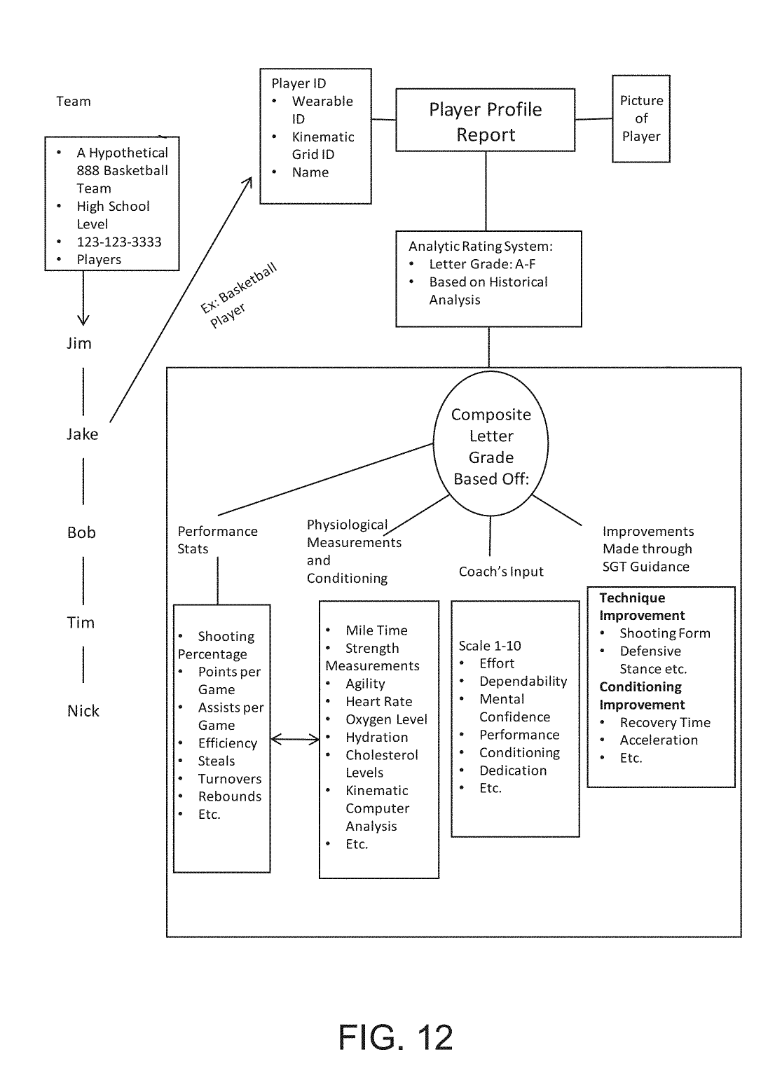

FIG. 12 is a block diagram illustrating an example of analytics and reporting system for an individual player 140. A basketball team exemplified in 401 plays at high school level and is composed of player Jim, Jake, Bob, Tim and Nick. Player profile report 402 (which might be generated by the analytics module 110 and stored in the sports analytics database 106) consists of picture of the player 403, player ID 404 including wearable ID, kinematic grid ID and name as well as SGT analytic rating system 405 etc. Letter grade rating (A-F) 406 is based on historical analysis of performance statistics, physiological measurements and conditioning, coach's input, improvements made through SGT, etc. Performance statistics includes, but not limited to shooting percentage, points per game, assists per game, efficiency, steals, turnovers, rebounds etc. Physiological measurements and conditioning includes, but not limited to mile time, strength measurements, agility, heart rate, oxygen level, hydration level, cholesterol level, kinematic computer analysis, etc. Coach's input includes, but not limited to effort, dependability, mental confidence, performance, conditioning, dedication, etc. Improvements made through SGT include, but not limited to technique improvement and conditioning improvement, etc.

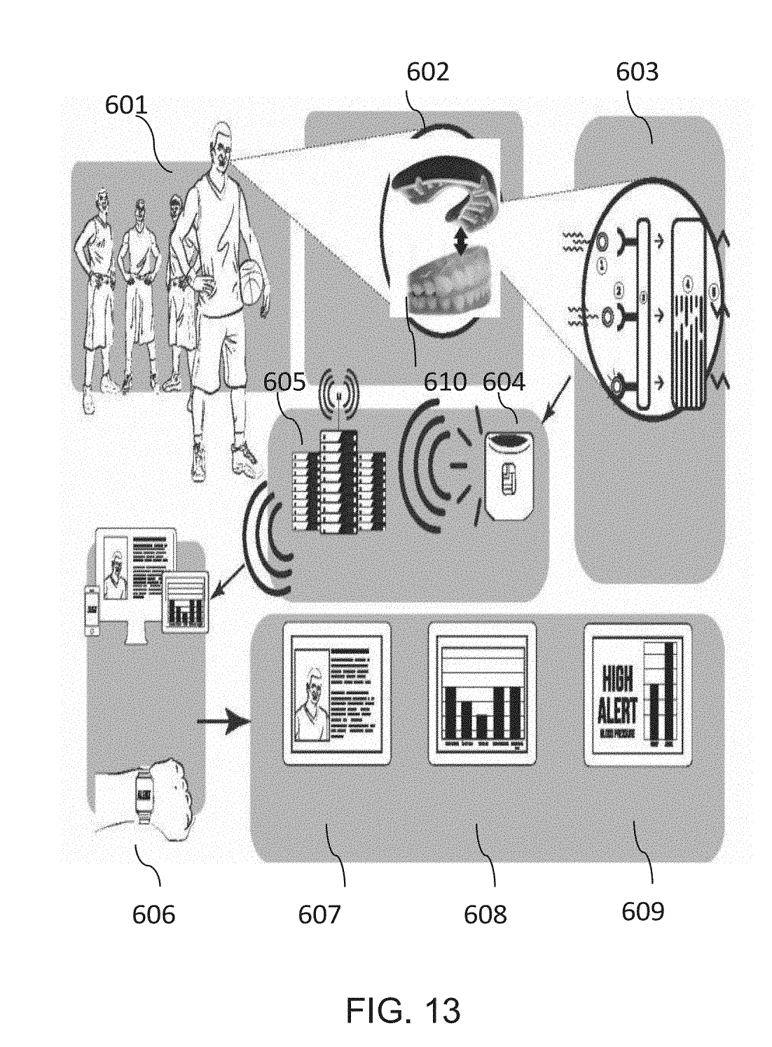

FIG. 13 is a diagram illustrating how the sports analytics system 100 generates sensor data based on internal devices 144 such as sensors in the players 140 oral cavities. Players 140, here exemplified by basketball players 601, can have sensors attached to their teeth, e.g., through an orally inserted device, or any dental device such as a retainer, partial guard, etc. or a combination of an orally inserted device and an accessory device such as a mouth guard, which could be coupled, fitted, attached, etc. to a partial guard or partial retainer 610, etc. The sensor 603 can detect any biologic, biologically relevant molecule, temperature, blood pressure, pulse rate, blood oxygen level, respiration rate, accelerometer, gyroscope, etc. In some situations, biosensors for heart rate, blood oxygen levels, etc. could be placed on the helmet or other head/face gear because these values from the central cardiovascular system might be required, and these could be measured from the carotid artery or its immediate branches. Biosensors or cameras could be placed on helmet parts or other head/face gear near or on the nose to get more accurate respiration rates. SGT devices could collect blood from bleeding due to gum disease, oral trauma and injury, testing, teeth and gum cleansing such as flossing, water pick, blushing, anything that causing or induce bleeding, pin-prick, etc. SGT device could be inserted in the oral cavity to be bathing in the blood to measure blood glucose levels, blood composition, blood chemicals, medication, etc. As needed, the information or signal can then be transduced, amplified, and processed 603, 2-4. The resulting signal can be transmitted through a RFID tag 603, 5, to an RFID reader on a wearable external device 142 such as an accessory, helmet, jewelry, wristband, clothing, or on other user devices 152 such as a smart phone, or others on, in or around the player, exemplified here by a smart wrist band 604. The wearable sports system can also include a RFID tag reader placed within or in proximity to any part of an oral cavity. The signal can then be bi-directionally transmitted to the coach 605. Not shown in the figures, but discussed herein, the smart wristband can also transmit signals from sensors on other locations on the player, equipment sensors 160 and sensors on other inanimate objects such as a smart ball, hoop, etc. around the player, environmental sensors 182 and also with other players 140 on the team. The information transmitted through the smart wrist band to the secure server can be through WiFi, Bluetooth, GPS, NFC, or other wireless methods, and in the absence of immediate conductivity, the information can be temporarily stored in the smart device as explained elsewhere herein 604. The secure server can bi-directionally transmit alerts to pre-selected user devices 152, such as smart phones, iPad, computers, etc. operated by personnel such as the player 140, coach, physician, or others chosen by the player, coach, etc. 606. The alerts can be transmitted when there are deviations from preset range values placed in the system for a biosensor and can also be of varying degrees and tiers as aforementioned. Also, as mentioned elsewhere herein, the physiological data can be viewed for an individual or collectively as a team and can be viewed in different formats such as, e.g., graphs, histograms, or pie-charts. Various screens can show or verbally narrate, e.g., via a talking computer, various information such as different comparatives with other players of a different or the same team, with comparisons made based on different sizes, ages, weights, gender, etc. or with a player or team's own previous history 607-609.

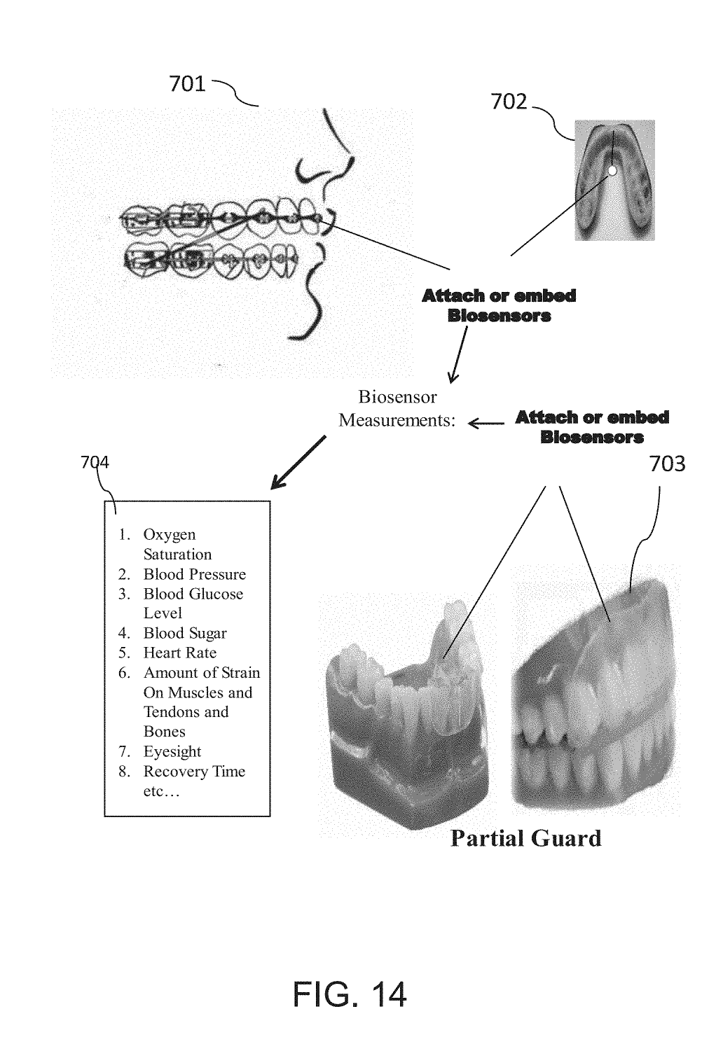

FIG. 14 is a diagram illustrating how the sports analytics system 100 generates sensor data based on internal devices 144 such as smart mouth guards. Internal devices 144 such as unfixed dental devices 701 are defined as ones not permanently attached to the jaw bone, but as possibly attached to the gum or teeth. Similarly, temporary biosensor mouth guards 702 and 703 have a generally shortened life span compared to fixed devices, but they may be placed in the oral cavity for from several minutes to several months (but typically are not designed for placement, e.g., for several years). As previously described, biosensors are optionally attached to or embedded in these devices. These biosensors could be custom-made by 3D printing. Biosensor physiological measurements 704 include, but not limited to, oxygen saturation, blood pressure, blood glucose level, blood sugar, heart rate, lactic acid build up, body temperature, hydration, amount of strain on muscles and tendons and bones, cholesterol levels, eyesight and recovery time etc.

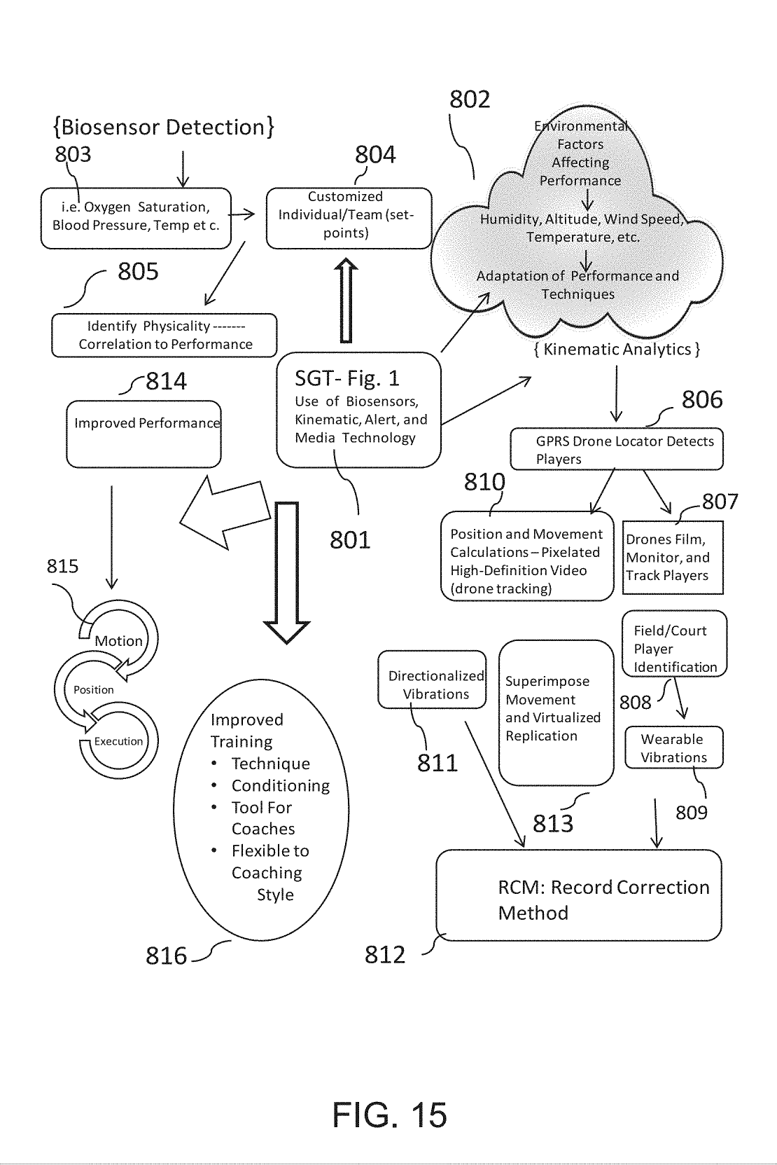

FIG. 15 is a diagram illustrating how the sports analytics system 100 integrates Sports Guidance Technologies to generate guidance information. As previously described, users 150 such as coaches and players 140 can use a variety of integrated sensor elements 410 such as biosensors, kinematic, alert and media technology to analyze all the factors that play into performance, thus improving performance 801. Environmental factors such as humidity and altitude etc. can have impacts on performance 802. The SGT devices such as the internal devices 144 and/or external wearable devices 142 generate sensor data indicating how these environmental conditions can alter performance levels and physiological characteristics within players 140, and the analytics module 110 generates guidance information based on the sensor data, the guidance information providing, for example, adaptations or adjustments in a player's 140 techniques or preparation in order to minimize the negative effect that some environmental factors may have on a player's execution during a competition. Sensor elements 410 such as biosensors are also integrated into the SGT devices 803. Once certain physiological attributes such as temperature, heart rate, or blood pressure etc. is identified within a player 140, users 150 such as coaches and trainers can then set optimal set points for players 140 (804). For example, in order for a player 140 to perform at his or her best, their physiological attributes such as temperature can't be too low or too high. So, their SGT devices will detect if the player's 140 physiological attributes go beyond or below a certain point according to the set points that coaches and trainers 150 have prescribed, and then immediately alert the coach or trainer through the user devices 152. This can effectively reduce the possibility of injuries and damage to body functions. The SGT sensors also analyze the performance statistics of a player 140 along with their physiological data 805. As a result, the SGT device can identify how the physiological conditions of a player 140 can directly impact the performance of a player during a competition, and can also provide different ways for players to increase their health, which ultimately leads to better performance. During training, the system 100 can also integrate an aspect of kinematic analysis to improve not only performance, but also team chemistry. Autonomous mobile devices 190 such as GPRS drone locators can be placed in the sports environment 180 such as the practice vicinity 806, and can film, monitor, and also track each player 140 on the field through the external wearable devices 142 that the player 140 puts on 807. In addition, the drones 190 can be set to identify a player 140 of where another certain player on the field/court is, through the player identification of the wearables that players have on 808. When a drone needs to alert a player of where another player is on the court, the feedback elements 412 such as vibration units within the external wearable devices 142 of players 140 will vibrate. The location and strength of the vibration will alert the player of another set player's position on the field so that a play can be made through these 2 players; thereby, improving the chemistry between the two players 809. During individual based skills training, the analytics module 110 can also identify the position and movement of the player 140 while he or she goes through certain exercises by means of kinematic identification and computer pixilation based on sensor data generated by the external wearable devices 142. After the precise movements of the player 140 are tracked, the drones 190 and analytics platform 102 can compare the movements of the player 140 to the precise movements and techniques of a professional sports player 810. If a certain movement proves to be inaccurate, then the analytics module 110 can send directionalized vibrations to the player 140 via the external wearable devices 142 of the player 140 and also suggest corrections to a player's movement, positon, and technique 811 and 812. This correction method can be known as the Record Correction Method (RCM). Another possibility for personal training with the SGT device is to superimpose the movements of a player 140 and virtualized players and their movements for a more interactive and effective training scenario 813. Every single environmental, physiological and kinematic (for example) can be analyzed by the analytics module 110 as it correlates to performance, so that players 140 and coaches 150 can better understand the relationship between these factors and performance 814; thereby, having a better understanding of not only maximizing performance, but also keeping performance at a peak level for the longest period of time possible for each player 140. The player's motion, position during competitions, and execution will all be improved, while training techniques and conditioning can also be refined 815. This is meant to be a flexible tool for coaches 150 to use as a part of their training program in order to maximize the effectiveness of training as well as performance 816.

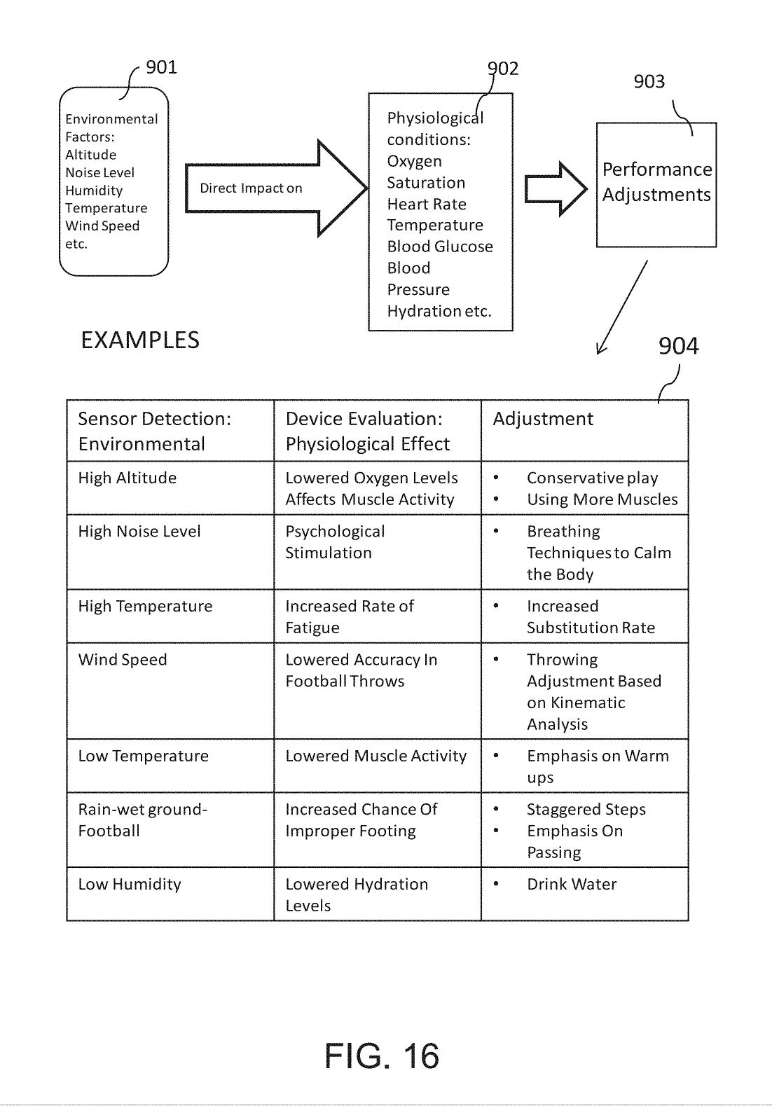

FIG. 16 is a diagram illustrating how the sports analytics system 100 integrates external environmental factors. Environmental factors 901 including altitude, noise level, humidity, temperature and wind speed, etc., can have direct impact on physiological attributes including oxygen saturation, heart rate, temperature, blood glucose, blood pressure and hydration etc., which results in performance adjustments 903 as detailed in 904, including more conservative play, using more muscles, breathing techniques to calm the body, increased substitution rate, throwing adjustments based on kinematic analysis by the analytics module 110, emphasis on warm ups, staggered steps, emphasis on passing, and drinking more water, among other examples.

For examples, when the sensor data generated by the environmental sensors 182 indicate high altitude, and the sensor data generated by the sensor elements 410 such as biosensors in the internal devices 144 detect lowered oxygen levels affecting muscle activity, the analytics module 110 may generate guidance information suggesting more conservative play and using more muscles etc. Similarly, when a high noise level is indicated by the sensor data generated by the environmental sensors 182 and psychological stimulation is detected based on the sensor data from the biosensors, the analytics module 110 may recommend breathing techniques to relax and calm down the body. In another example, as an increased rate of fatigue is determined by the biosensors at high temperatures, the analytics module 110 may generate guidance information suggesting an increase in a substitution rate for the player 140. In another example, wind speed reduces the accuracy in football throws, therefore the analytics module 110 can suggest throwing adjustments based on kinematic analysis of sensor data generated by the environmental sensors 182. Low temperatures lead to lowered muscle activity, in which case the analytics module 110 may instruct more emphasis on warm ups. Wet ground resulted from the rain increases the chance of improper footing during football game. Upon detection of wet ground based on the sensor data, the analytics module 110 might recommend staggered steps and focusing on passing. As low humidity lowers hydration levels of players 140, the analytics module 110 might suggest drinking more water, based on the sensor data.

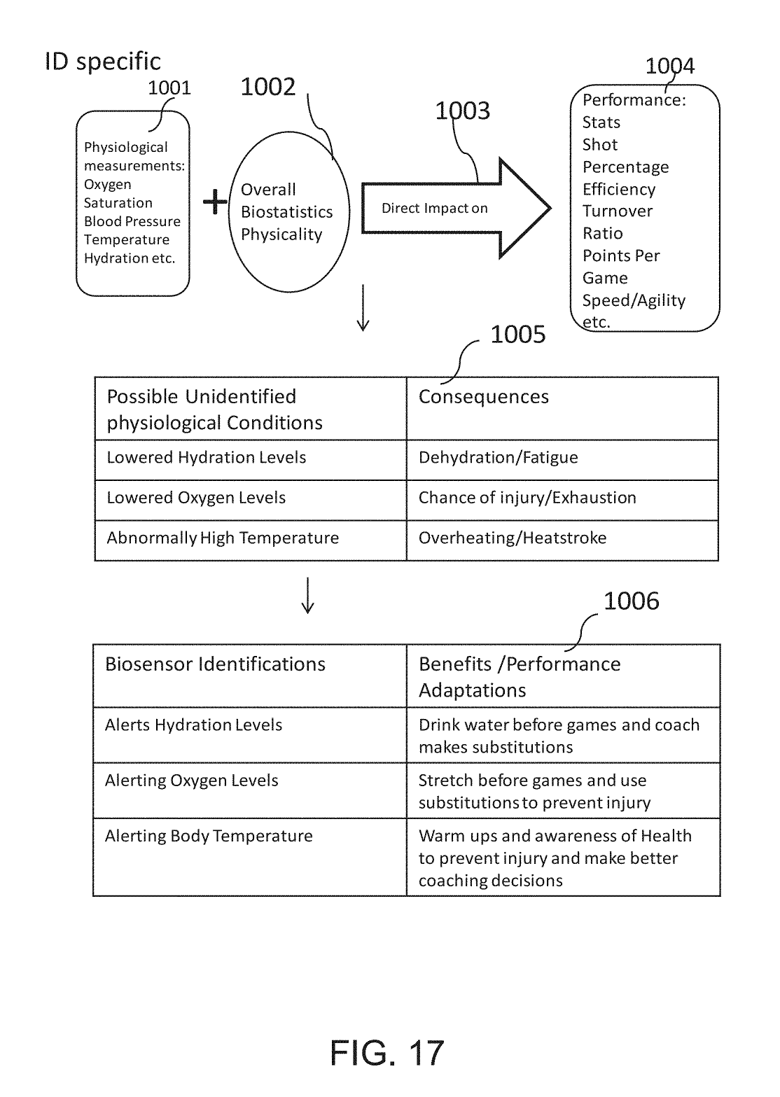

FIG. 17 is a diagram illustrating how the sports analytics system 100 analyzes physiological measurements in relation to performance. Physiological measurements 1001 including oxygen saturation, blood pressure, temperature and hydration etc. along with overall biostatistics physicality 1002 can have direct impact 1003 on performance statistics 1004 including shot percentage, efficiency, turnover ratio, points per game, speed and agility etc. Sport injuries including fatigue, exhaustion and heatstroke, etc. could be resulted from some unidentified physiological conditions such as lowered hydration levels, lowered oxygen levels and abnormally high temperatures, etc. 1005. Sensor data generated by sensor elements 410 such as biosensors of the internal devices 144 can be applied by the analytics module 110 to generate alerts regarding hydration levels, oxygen level and body temperature, etc. As a result, performance adaptation can be planned which includes drinking more water before games, substitutions, stretch before games and warming up, etc. Biosensors which provide real-time alerts on the health conditions can effectively prevent injury and help coach make better decisions 1006.

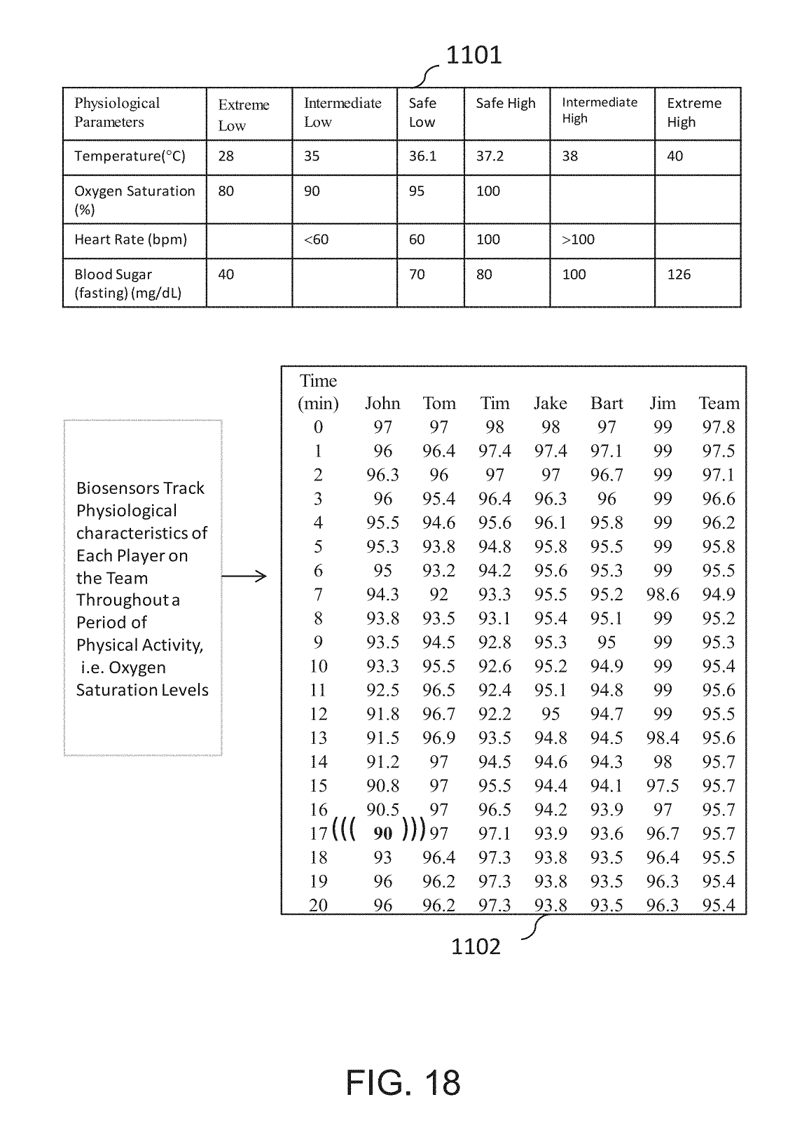

FIG. 18 is an illustration of exemplary sensor set points, sensor data collection and alert and report generation. Sensor predetermined set points for physiological parameters such as temperature, oxygen saturation level, heart rate and blood sugar etc. are listed in 1101. These set points might be based on input received (e.g. from coaches 150) via the user devices 152 and stored in the sports analytics database 106 associated with the teams and players 140. The analytics module 110 monitor oxygen saturation level of each player 140 on the team based on sensor data from sensor elements 410 such as biosensors of the internal devices 144 throughout a period of physical activity is shown in 1102. An alert 1102 is generated and transmitted to the user devices 152 and/or the external wearable devices 142 (to be communicated to the player 140 via the feedback element 412) when the oxygen saturation level from player 140 John drops to 90%, according to the sensor set point for intermediate low alert as listed in 1101.

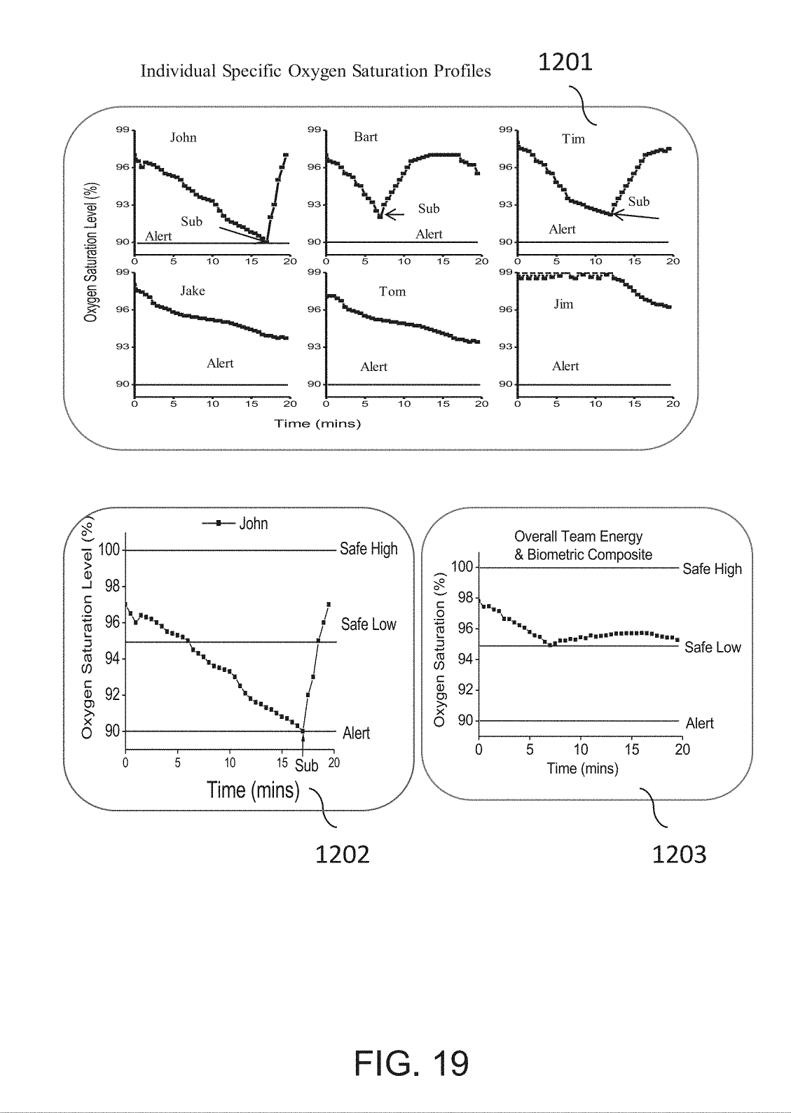

FIG. 19 is an illustration of exemplary graphical representations of data collection (including, for example, sensor data and/or analytics data generated based on the sensor data), indicating when alerts and reports might be generated by the analytics module 110. The profiles of oxygen saturation level for each individual player 140 (John, Bart, Tim, Jake and Tom) during a basketball game are presented in 1201. Oxygen saturation level of 90% is set as an alert limit and stored in the sports analytics database 106. Intense physical activity in the game causes decreased oxygen levels for all the players 140, although the extent of reduction varies. Seventeen minutes into the game, John's oxygen saturation level drops to the alert limit 90%, so the coach 150, who might be monitoring the graphical representations via the GUI 154 of the user device 152 or who might receive an alert generated by the analytics module 110, replaces him with a substitute. As a result, John's oxygen level starts to recover 1201&1202. For a different player Bart, only seven minutes into the game, the coach 150 notices that his rate of oxygen decrease is much faster compared to other players 140 in the team, indicating suboptimal physical conditions. So the coach 150 immediately replaces Bart even before his oxygen level hits the alert limit 1201. Subsequently, Bart's oxygen level recovers. And when the coach 150 sees Bart's oxygen level reach and maintain at a high level for some time, he puts Bart back to the game in 17 minutes as a substitute for John. A similar scenario happens to Tim who is replaced by Jim in twelve minutes but does not return to the game due to his slow recovery as indicated in the graphical representations. Jake and Tom play the whole game since their rates of oxygen decrease are slow and both performances are strong. Jim doesn't play at the beginning, so his oxygen level is kept constant until he substitutes Tim in 12 minutes. According to the sensor set points, “safe high” and “safe low” levels for oxygen saturation are plotted along with “alert limit” as shown in 1202&1203. Overall team energy and physiological composite are plotted in 1203. Compared to the big fluctuations of oxygen level in each individual player, the change in the overall team composite is relatively small and the average maintains above the “safe low” level. Even at the beginning of the game, the reduction in oxygen level for the team is much slower. By substituting players at three critical moments (at seven minutes, twelve minutes and seventeen minutes), the team average oxygen level manages to remain at competitive levels throughout the game. An inflection point occurs when a player's 140 oxygen level stops decreasing and starts to recover after he is replaced by a substitute as shown in 1201. Thus the inflection point can be used to track substitution of the players 140 during the game.

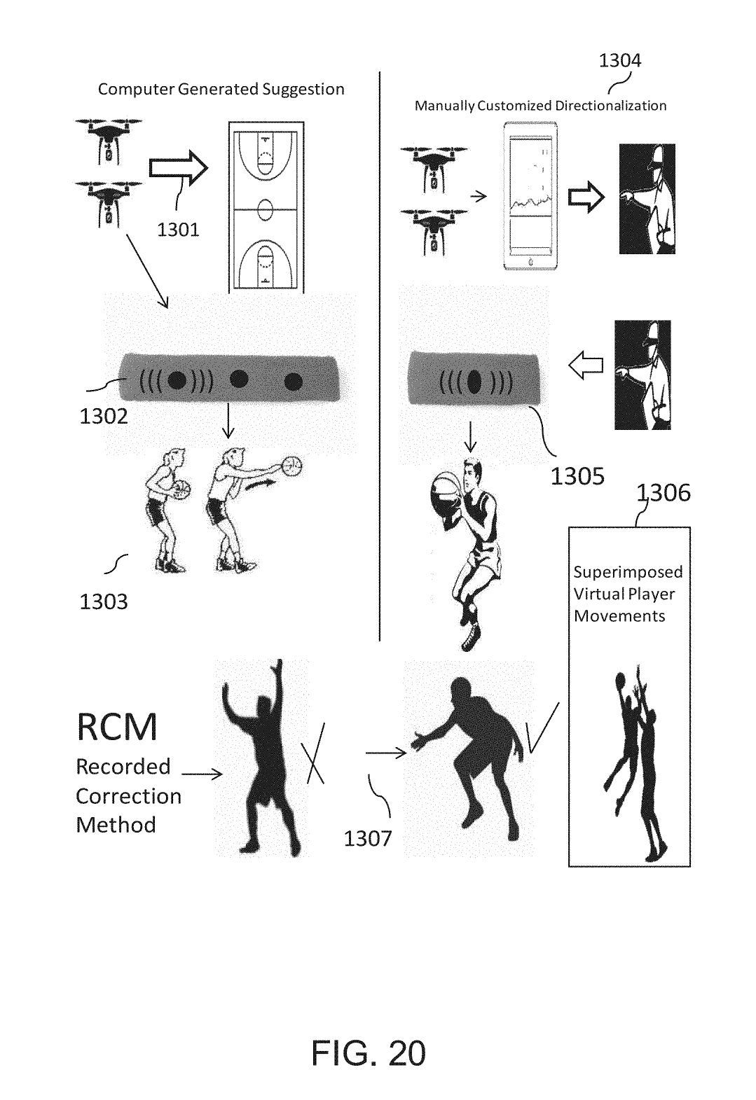

FIG. 20 is a diagram illustrating how the sports analytics system 100 analyzes kinematic factors to maximize performance through the kinematic identification, analysis, and directional guidance of each player 140 based on sensor data generated by the external wearable devices 142 and internal devices 144 of the players 140. In 1301, the autonomous mobile devices 190 such as drones generate image data depicting the players 140 in the sports environment 182 (e.g. on the field), tracks the players 140, and sends alerts to the players 140 via the feedback elements 412 of the external wearable devices 142 of the players 140. Vibrations in different locations of the external wearable devices 142 are utilized to alert players 140 where another certain player is on the floor 1302. By doing so, it increases the chance to score for a certain team and ultimately improves team chemistry. For example, in a basketball game, the feedback element 412 of the external wearable devices 142 associated with the left arm (e.g. left arm sleeve of the player's 140 jersey or armband) generates a vibration based on instructions from the autonomous mobile device 190 and/or the analytics module 110. As a result, the player 140 knows that there is another player 140 on his left that he can pass to and possibly get a shot off. By using vibration oriented communications, team chemistry among players 140 is thus improved. In 1304, the kinematic information of each player 140 that is tracked can also be sent (e.g. as a high definition video) in real time or near real time to the user devices 152 operated by the coaches 150. Coaches 150 are also able to enter input via the GUI 154 of the user device 152 indicating setting certain sensors and vibrations to certain players 140. For examples, in basketball, coaches 150 can specifically set vibration alerts between the point guard and a center so that the point guard can be alerted of where the center is. As a result, the point guard may then have the information he needs to get the center the ball for him to get a wide open layup 1305. The sports analytics system 100, for example via the analytics module 110, can also superimpose the movements of a player 140 onto virtualized players and their movements for a more interactive and effective training scenario 1306. For example, training with a virtualized player replication that has superimposed movements can be used to correctly guide the player 140 during training so that a comprehensive learning environment can be created between a virtualized player and the player 140 who is training. As a result, players 140 learn what to do in certain game situations 1306. Precise movements of the player 140 can be tracked via the sensor data generated by the external wearable devices 142, and the autonomous mobile devices 190 and/or analytics platform 102 then compare the movement of the players 140 to the precise movements and techniques of professional sports players. If a certain movement proves to be inaccurate, then the autonomous mobile devices 190 and/or the analytics platform 102 send directionalized vibrations to the players 140 via the feedback elements of the external wearable devices 142 of the players 140, suggesting corrections to the players' movement, position and technique. This correction method can be known as Record Correction Method (RCM) 1307. For example, if the defensive stance of a basketball player 140 is off balance, the Record Correction Method not only alerts the player 140 that his form is off, but can also guide him to have the defensive form of a professional basketball player through directionalized vibration that can be paired with coaching as well.

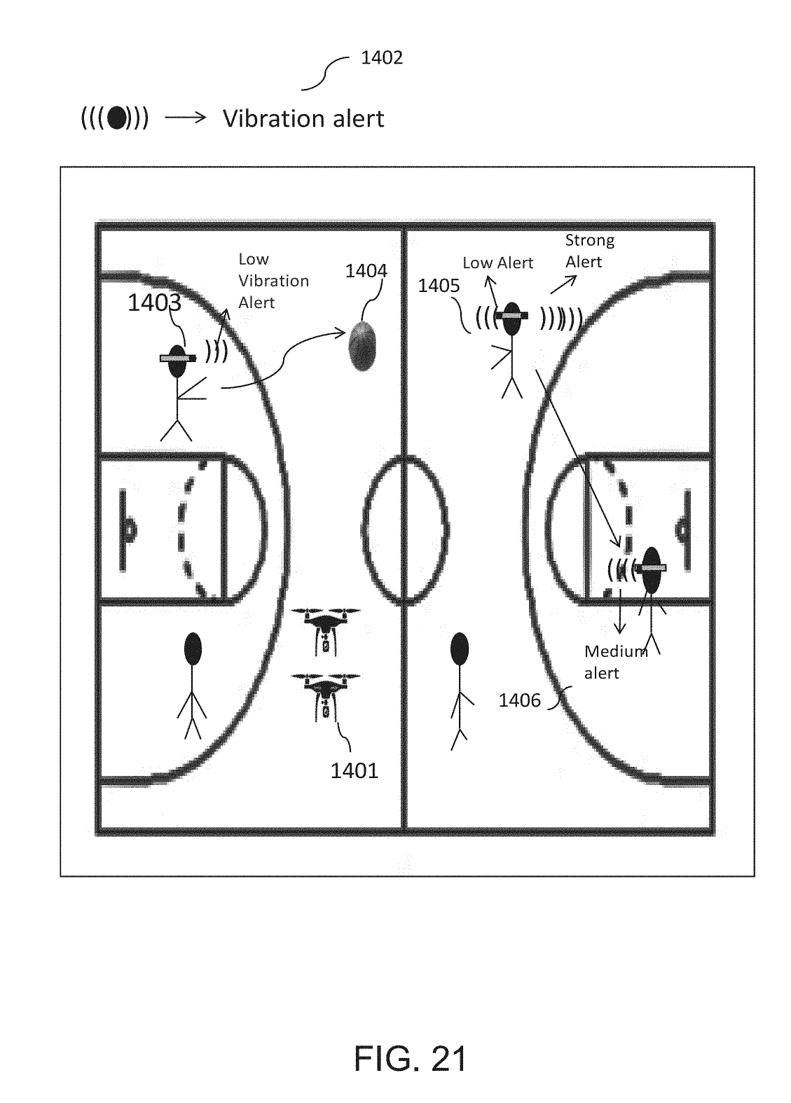

FIG. 21 is an illustration of how the sports analytics system 100 analyzes kinematic factors to maximize performance as described with respect to FIG. 20. In the illustrated example, the scenario is in the context of a basketball game where there are five offensive players 140 on the field 180. In 1401, drones 190 are used to monitor the court and track players 140 through the external wearable devices 142 of the players 140. In this instance, vibrations as indicated by the exemplary symbol in 1402 on each player's 140 external wearable devices 142 (e.g. delivered via the feedback elements 412) illustrate how the system can not only direct players 140 into making the right plays that ends in scoring for the team, but also improves team chemistry as well. The location of the vibration on the external wearable devices 142 is what determines the general location where the player is as well as the general angle of which the pass of the basketball should be directed towards. The strength of the vibration determines the distance as well as the velocity in which a player 140 has to throw the basketball in order for the ball to get to the next player 140 most effectively. Low vibrations represents the distance of one player 140 to another player 140 is long while a stronger vibration means the distance between 2 players is shorter. In this specific example, the player gets the rebound from one side of the basketball court and looks down the floor in 1403. The drones 190 also detect the player who got the rebound. Immediately after, drones detect another open player farther down the court that is sprinting down the floor. Once the open player is identified as the smartest and most effective play, a low vibration in the frontal location of the player with the ball's headband alerts the player that he needs to throw the ball at a 90 degree angle east 1404 with a high velocity in order to get it to the next most effective open man 1405.

The open man 1405 on the other end of the floor will also get a vibration that alerts him that a pass is coming his way. Once he receives the pass, another vibration on the left side of his headband alerts the player that there is another open man 1406 right by the basketball hoop that can score easier than he will. The strong consecutive vibration 1405 tells the player that he is close to the open man 1406, which means he needs to throw a pass that is at 135 degrees southeast which is a quick zip pass in order to most effectively transfer the ball to the open man 1406 by the basket. Finally, the open man 1406 receives a medium vibration that alerts him that a ball is coming his way for him to score. This set play identified by the wearable sensors ultimately results in a play for the team to score and improves their on court knowledge of basketball plays as well as their team chemistry with one another.

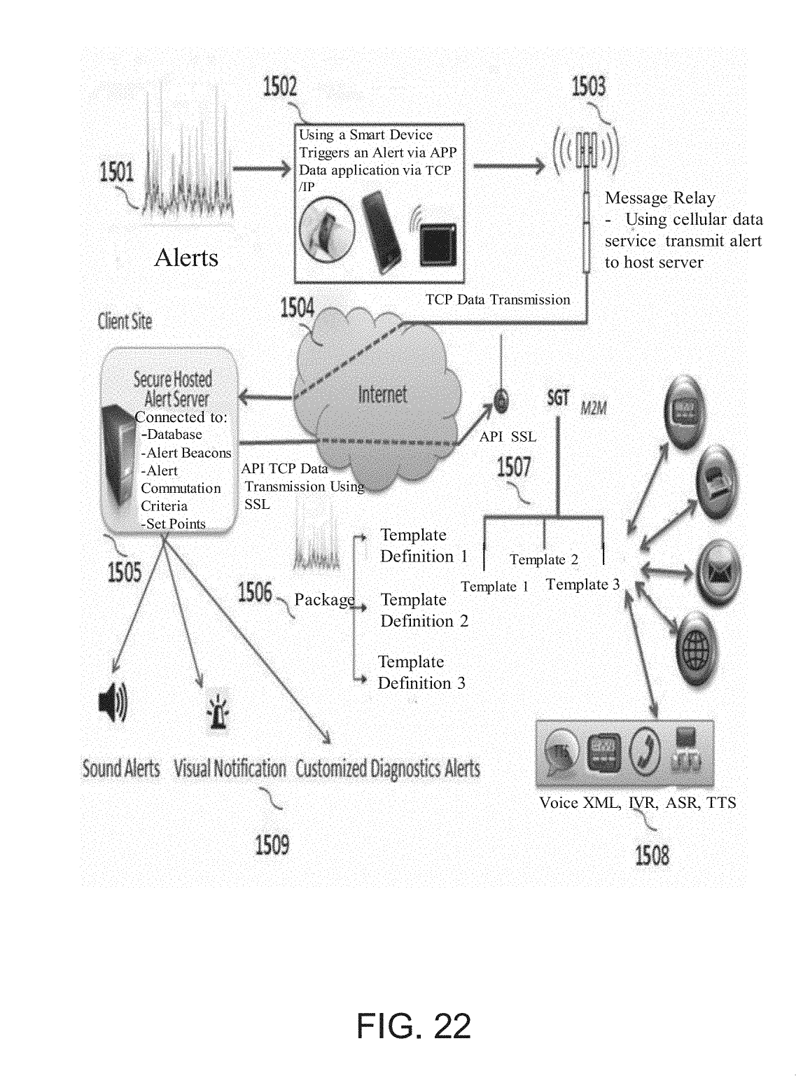

FIG. 22 is a diagram illustrating how the sports analytics system 100 functions as a fully integrated diagnostic and performance measurement system. 1505 represents a secure host server such as the analytics platform 102 which can be implemented and utilized by one or more individuals, one or more animals, or one or more organizations. This can include a privatized internal server host and subsystems as well as one or more external hosted alert servers. A plurality of collective data can be derived from sensor data from several SGT oral measurements including, but not limited to, the integration of any type of external wearable device 142 and/or internal device 144. The biosensor data from all devices of the sports analytics system 100, including all external wearable devices 142 and/or internal devices 144, whether smart or not smart, and all RFID readers, all can be examined and analyzed (e.g. by the analytics module 110) in order to determine the degree of an alert (low, medium or high) being dispatched through various templates 1507 referred to today as cloud networks which includes all forms of user devices 152 such as smart devices, one or more pagers, SMS, Faxes, emails, GIS mappers, beacons (XYZ) telephones, PSTN devices 1508 (Voicemail, IVR, ASR, TTS), satellite phones and other forms of communication. The alert can be dispatched to any computer-aided device or emergency dispatch if the SGT device detects higher than average or abnormal metabolic ranges, for example. The SGT device can use one or more templates to help delineate these physiological ranges as exemplified by 1501. 1506 exemplifies the packaging of biosensor parameters as defined (Definition 1, Definition 2, Definition 3 . . . ) by users 150 such as the individual, coach, team and organization etc. In addition, the alerts can be streamed, packeted or stored on the server (e.g. in the sports analytics database 106) or on the person(s) or animal(s). Alerts can be represented through preset criteria notification icons converted to SMS, SMS or icons converted to voice alerts, visual notification, touch (vibration) auditory notification and customized through one or more algorithms and diagnostics and secure databases, servers and networks can be used. In addition, bi-directional or multi-dir ectional 1504 API/TCP data, i.e., SSL (128-Bit) data transmissions can use SSL and a message relay using cellular data services 1503 transmitted through one or more host servers. Data application can be the triggering of the alert as previously described, and can be automated (M2M), manual or a combination of both. SGT alerts can also be combined with APP public general alerts for one or more geographies.

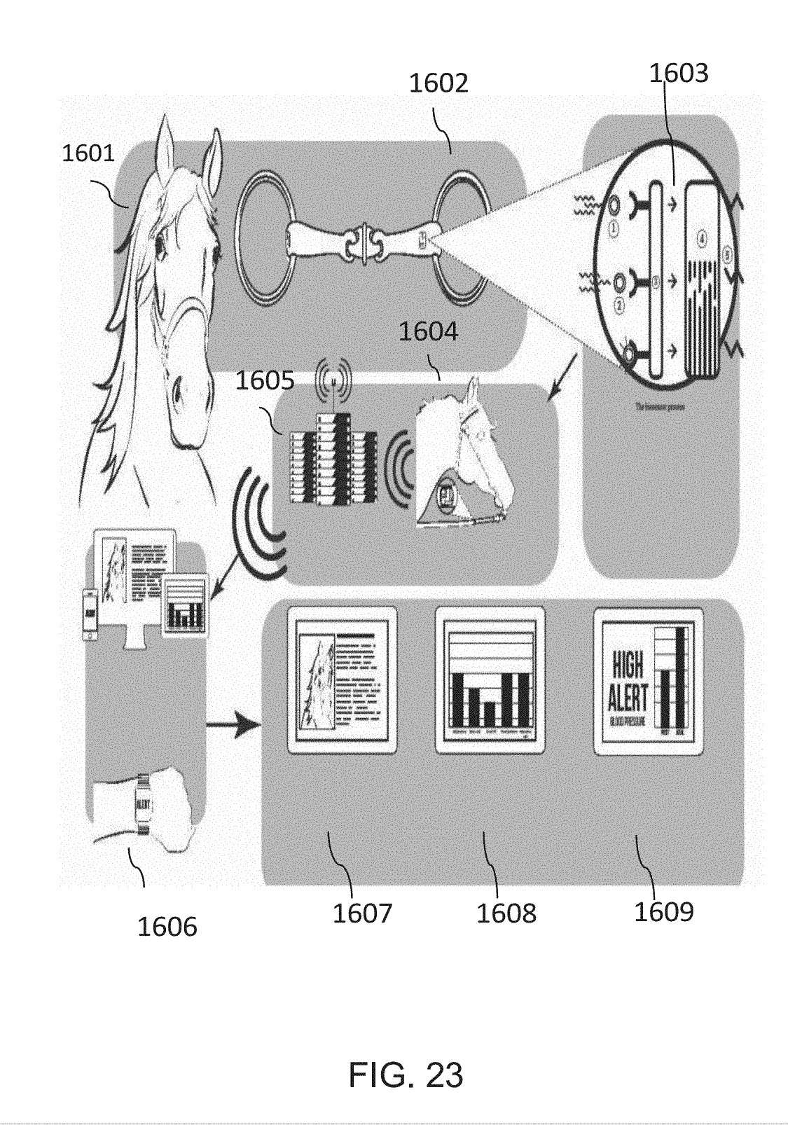

FIG. 23 is a diagram illustrating how the sports analytics system 100 analyzes sensor data generated for an animal player 140, exemplified here by a race horse. In 1601, the race horse 140 wears or has implanted wearable external devices 142 and/or internal devices 144 such as a mouth-bit, bit-guard, bit-gag, lip-strap, or other dental device 1602. The devices are equipped with sensor elements 410 such as biosensors. In 1603, the sensor elements 410 detects any biologic, biologically relevant molecule, temperature, blood pressure, pulse rate, blood oxygen level, respiration rate, as well as motion via a gyroscope, accelerometer, etc. Sensor elements 410 of the devices might collect blood from bleeding due to gum disease, oral trauma and injury, testing, teeth and gum cleansing such as flossing, water pick, blushing, or anything that causes or induces bleeding such as a pin-prick, etc. Internal devices 144 are inserted in the oral cavity to be bathing in the blood to measure blood glucose levels, blood composition, blood chemical, medication, etc. As needed, the information or signal can then be transduced, amplified, and processed 1603. The resulting signal is transmitted through, for example, a RFID tag 1603 to an RFID reader on, e.g., another accessory such as an external wearable device 142 attached to the horse 140, including a collar, rein, saddle, or on a horse-rider or jockey, or on user devices 152 such as the jockey's smart phone, or others, on, in, or around the horse, which could read the sensor data from the biosensors located in the bit when in the horse's mouth, exemplified here by an external wearable device 142 such as a smart rein 1604. In some situations, biosensors for heart rate, blood oxygen, and other sensors such as a gyroscope, accelerometer, inertia-sensor, tracking sensors, camera, video, microphone, speakers, etc. could be placed on the external wearable devices 142 for the horse such as, but not limited to, headstall, headgear, ear-poms, blinker hood, hackamores, noseband, cheese-band, bridle, blinders, winkers, ornaments such as phalerae and sallongs, etc. Various values which integrate the oral bit guard data from the central cardiovascular system could assist in measuring both performance and health of the horses 140. In another example, external wearable devices 142 such as the blinker hood or nose-piece, or devices attached to the horse's nose or other facial parts, with sensor elements 410 such as biosensors or cameras, detect accurate respiration rates. In addition, a heart-monitoring device, heart-rate, or respiration monitoring device can be attached to the saddle or other horse equipment attached to or associated with the horse. The horse's heart rate can also be monitored via equipment sensors 160 and/or environmental sensors 182 such as a manure catcher, or other external wearable devices 142 such as a diaper such that the sensors are under the tail at the tailbone. The heart rate can also be measured by wireless biosensors on horse's leg or other body part. To measure performance, external wearable devices 142 equipped with sensor elements 410 such as accelerometers, gyroscope, inertia-sensors, etc. can be placed at various parts of a horse's body, such as its legs, neck, torso, etc. An external wearable device 142 and/or internal device 144 including an RFID tag reader can also be placed within or in proximity to any part of an oral cavity, temporarily or permanently. Not shown in the figure, but disclosed elsewhere herein, similar to an application for an athlete, the smart horse-rein, e.g., can also communicate a signal from sensors on the horse and other inanimate objects around the horse and from other horses. In 1605, the signal can then be bi-directionally transmitted to a secure server such as the analytics platform 102. In 1605, the information transmitted through the smart horse-rein, e.g., to the secure server can be through WiFi, Bluetooth, GPS, NFC, or other wireless methods, and in the absence of immediate conductivity, the information can be temporarily stored in the smart device via the nonvolatile memory 408 as previously described. In 1606, the secure server can bi-directionally transmit alerts to pre-selected user devices 152 devices, such as smart phones, iPad, computers, etc. operated by users 150 such as the owner, veterinarian, jockey, or others chosen by the owner. The alerts can be generated and/or transmitted (e.g. by the analytics module 110) when there are deviations from preset range values (e.g. stored in the sports analytics database 106) for a biosensor and can also be of varying degrees and tiers as aforementioned. Also, as mentioned elsewhere herein, the analytics module 110 can generate the physiological data and visualizations of the physiological data in different formats such as, e.g., graphs, histograms, or pie-charts. In 1607, 1608, and 1609, various screens of the GUI 154 of the user devices 152 can show or verbally narrate, e.g., via a talking computer, different information such as different comparatives with other race horses of different, similar or the same sizes, ages, weights, gender, etc. or with the horse's own previous history.

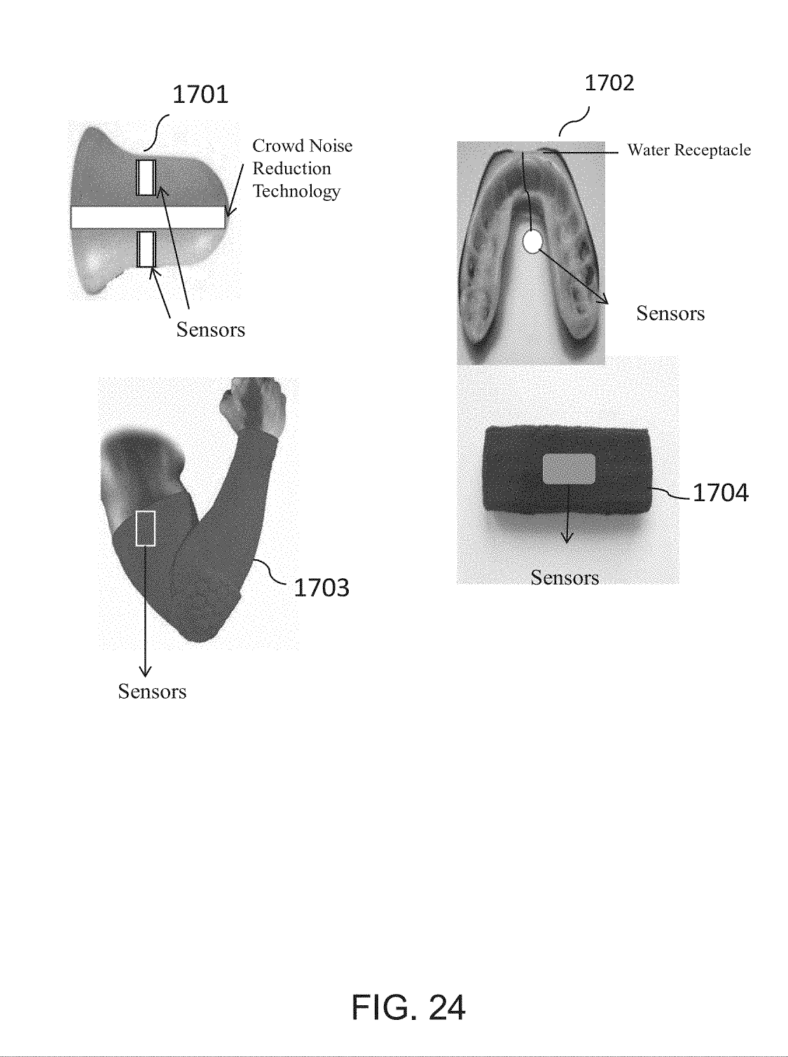

FIG. 24 is an illustration of examples of external wearable devices 142 and internal devices 144 of the sports analytics system 100, as they might be integrated for performance measurement. In 1701, the external wearable device 142 is a smart earbud, which includes crowd noise reduction technology to decrease noise level from the environment and allows oral communications among coaches and players to be heard more clearly. The smart ear bud might include sensor elements 410 such as biosensors measuring temperature, heart rate, blood O2, as well as accelerometers, gyroscopes and others can. Similarly, these same sensor elements 410 might be included in other external wearable devices 142 such as a smart arm band as illustrated in 1703, a smart head band as illustrated in 1704, and internal devices 144 such as a smart mouth guard or retainer as illustrated in 1702. All the external wearable devices 142 and/or internal devices 144 placed in all parts of the body can be integrated by the sports analytics system 100 for performance measurements.

Also, for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. Signals and corresponding nodes or ports may be referred to by the same name and are interchangeable for purposes here.

It should be understood that the steps of any exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

As used herein in reference to an element and a standard, when used, the term “compatible” means that the element communicates with other elements in a manner wholly or partially specified by the standard, and would be recognized by other elements as sufficiently capable of communicating with the other elements in the manner specified by the standard. The compatible element does not need to operate internally in a manner specified by the standard.

It will be further understood that various changes in the details, materials, and arrangements of the parts which have been described and illustrated in order to explain the nature of this invention may be made by those skilled in the art without departing from the scope of the invention as expressed in the claims.

What is claimed is:

1. A system comprising a device configured to be inserted or attached to an animal or human comprising a smart sensor receptacle for a sensor, the device further comprising one or more wearable sensors contained within or upon the receptacle, and at least one interface with a network configured to utilize the information obtained from the one or more sensors or from one or more platforms.

2. The system of claim 1 wherein one or more functions of the device is selected from the group consisting of providing sports function, health analytics, diagnostic analytics, performance analytics; integration of wearable sensors, health-devices, sports and performance sensors on inanimate objects and sports equipment; sports gear, clothing, stadium, ballpark, park; gym, gymnasium, arena, dome, bowl, circus, coliseum, colosseum; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, historical analysis, time stamped data, reporting and feedback, positioning, the integrated video can be synced with all wearables and other biosensors in order to produce computer-generated precise movement and greater precision or analytics.

3. The system of claim 1 wherein the system further comprises a database compilation of one or more players' performance entries.

4. The system of claim 1 wherein the system further comprises a database compilation of one or more players' physiological attributes.

5. The system of claim 1 wherein the system further comprises a database compilation of one or more players' kinematics.

6. The system of claim 1 wherein the system is further configured to analyze individual or team sports performance as it relates to various body components and sensors.

7. The system of claim 1 wherein the system is further provided with full connectivity and full server access.

8. The system of claim 1 wherein the system is further configured to provide an alerting signal when outside a predetermined set point.

9. The system of claim 1 wherein the system is further configured to operate as training and coaching network.

10. The system of claim 1 wherein the system further comprises physical tracking and precision field logistic software, a digital transactional communication interface and controls, navigation and operational guidelines configured to facilitate performance.

11. The system of claim 1 wherein the system further comprises software configured to provide athletic analysis, logistics, specialty location XYZ modules, date entry timestamp and input.

12. The system of claim 1 wherein the system is further configured to be customizable by a user in a sports practice or game mode to facilitate performance and optimal player health.

13. The system of claim 1 wherein the system further comprises a historical database of the animal or human as to one or more characteristics from which comparisons or analyses are to be made.

14. The system of claim 1 wherein the system further comprises means to optimize the play time of an individual player during a game or training.

15. The system of claim 1 wherein the system further comprises smart data compiler software configured to data stream information for use by the user to evaluate one or more players' performances when playing in a sport or requiring an athletic performance.

16. The system of claim 1 wherein said network is configured to carry out a functionality selected from the group consisting of signaling bi-directional transmissions to a secure server through one or more of WiFi, Bluetooth, GPS, NFC or other wireless means, temporarily storing information in the smart device, bi-directionally transmitting alert to pre-selected devices or pre-selected personnel.

17. The system of claim 1 wherein said network is configured to analyze a composite input of a plurality of team or group members.

18. The system of claim 1 wherein said network interfaces with a mobile device or apparatus.

19. The system of claim 1 wherein said network interfacing with the mobile device provides sensor information or analysis to a user.

20. The system of claim 1 wherein said network capable of utilizing the information obtained from the one or more sensors comprises one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

21. The system of claim 1 wherein information utilized by said network includes one or more of measurements of performance, measurements of health, measurement of energy level, measurement of physiological attributes, information obtained from sensors, kinematics information, information obtained from cameras, information from sensors inserted or attached to body parts, information from instruments used to measure performance, information received from sensors attached to or associated with inanimate objects and sports equipment.

22. The system of claim 1 wherein said network comprises means by which one or more sensors are activated by another sensor, device or remote controller.

23. The system of claim 1 wherein said network comprises means for integrating one or more wearable sensors with sensors attached to or associated with inanimate objects or sports equipment.

24. A method of training comprising providing a virtual presentation of one or more athletes for visualization by one or more users.

25. The method of claim 24 wherein said virtual presentation is configured to be three-dimensional profiles customizable by said user to facilitate performance.

26. The method of claim 24 wherein the method further comprises providing one or more data servers for the user to virtually display three-dimensional profiles of one or more bodies or limbs for precise movement and analysis.

27. The method of claim 24 further comprising providing a controller with the capacity to configure the database of one or more sensors and predetermined set points, scale, type of sport, athlete, individual energy alerting, team energy alerting, physiological computations, historical references, search engine and analytics.

28. The method of claim 24 further comprising providing an analytical processing capability comprising motion and performance comparison.

29. The method of claim 24 wherein said virtual presentation of one or more athletes comprises a holographic images and patterns of synced simulations.

30. A method of training comprising utilizing a network of wearable sensors to guide a player or teams.

31. The method of claim 30 wherein said network is configured to activate said wearable sensors to define said player's or teams' motions.

32. The method of claim 30 wherein said network is configured to correct said player's motor skills and make adjustments to optimize said player's or teams' performance.

33. The method of claim 30 further comprising utilizing vibrations on a player's wearable devices to perform directional guidance.

34. A method of training comprising utilizing artificial intelligence to determine an efficient motion for a player.

35. The method of claim 34 further comprising correcting or adjusting said player's motion through the wearable devices.

36. A method of training comprising utilizing robotics in a training or game to film, track or guide a player through the wearable devices.

37. A customizable tool kit or platform for building a wearable sports system to provide information, analysis or alerts for an animal, animals, human or humans, comprising a kit or platform of customizable components to meet the needs of a developer, consumer or user of the system, the components comprising at least one sensor inserted or attached to the animal, animals, human or humans, at least one receptacle configured to contain or receive the sensor, and at least one network unit configured to receive information, analysis or alerts from or transmit information, analysis or alerts to the at least one sensor and analyze, transmit, or both, the information, analysis or alerts obtained or received, wherein components for selecting the sensor receptacles, the sensors, and the network units are made available to the developer, consumer or user to construct or have constructed a wearable sports system configured to obtain or transmit information, analysis or alerts customized to meet the specific needs of the developer, consumer or user.

38. The tool kit or platform of claim 37 wherein a preselected set of kit or platform components is provided in the kit or platform together with instructions for building the desired system.

39. The tool kit or platform of claim 37 wherein the system is designed for a sports function, health analytics, diagnostic analytics, performance analytics; integration of body sensors, health-devices, nano-particles, sports and performance sensors on inanimate objects and sports equipment; sports gear, clothing, stadium, ballpark, park; gym, gymnasium, arena, dome, bowl, circus, coliseum, colosseum; customizable developers' tool kit for biosensors, sensors, performance, medical analytics, oral and systemic diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

40. The tool kit or platform of claim 37 wherein the tool kit or platform further comprises a software control system configured to authenticate, analyze and gather data to guide, enhance performance.

41. The tool kit or platform of claim 37 wherein the tool kit or platform further comprises a software control system configured to provide one or more of the functions of tagging, tracking, logging data regarding smart sports equipment, smart sensor wearables as it relates to sports movement.

42. The tool kit or platform of claim 37 wherein the toolkit or platform further comprises a software control system configured to provide one or more of the functions of facilitating secure communication, adjusting motor skills, permeating smart particles and materials, entering secure data points and data sets which assist in coaching, training and athletic performance.
